(19) United States
(12) Patent Application Publication
(10) Pub. No.: US 2012/0207719 A1
(43) Pub. Date: Aug. 16, 2012

(54) PANCREATIC ISLET-LIKE CELLS

(75) Inventors: Glenn E. Winnier, The Woodlands, TX (US); Brian S. Newsom, Spring, TX (US); Donna R. Rill, The Woodlands, TX (US); Jim C. Williams, The Woodlands, TX (US)

(73) Assignee: Opexa Therapeutics, Inc., The Woodlands, TX (US)

(21) Appl. No.: 13/428,651

(22) Filed: Mar. 23, 2012

Related U.S. Application Data
(63) Continuation of application No. 12/299,590, filed on Nov. 4, 2008, now abandoned.

(30) Foreign Application Priority Data
May 4, 2007 (US) PCT/US07/68303

(51) Int. Cl. A61K 35/12; A61P 3/10; C12N 5/071

(52) U.S. Cl. 424/93.7; 435/366

(57) ABSTRACT

The generation of pancreatic islet-like cells from isolated monocyte-derived stem cells (MDSCs) is provided. MDSCs may be differentiated into pancreatic islet cells by contacting the MDSCs with a differentiation factor or factors. Compositions comprising pancreatic islet cells and methods of using them are also provided.

### BACKGROUND

1. Field of the Invention

This invention relates to methods of generating pancreatic islet-like cells, compositions of pancreatic islet-like cells, and methods of using pancreatic islet-like cells.

2. Description of Related Art

Diabetes is a disease characterized by the failure or loss of pancreatic β-cells to generate sufficient levels of the hormone insulin required to meet the body's need to maintain normal nutrient homeostasis. There are two forms of diabetes; type 1 (juvenile) and type 2 (adult late onset). Type 1 diabetes is caused by the complete loss of pancreatic β-cells when the body's own immune system mistakenly attacks and destroys a person's β-cells. For type 2 diabetes the causes are far more complicated and poorly understood, the results of the disease are similar in that the β-cells fail to generate sufficient amounts of insulin to maintain normal homeostasis. The loss of insulin results in an increase in blood glucose levels and eventually leads to the development of premature cardiovascular disease, stroke, and kidney failure. Currently there is no cure for diabetes; however, daily injections of insulin can help regulate blood glucose levels. For these patients, frequent monitoring is important because patients who keep their blood glucose concentrations as close to normal as possible can significantly reduce many of the complications of diabetes, such as retinopathy (a disease of the small blood vessels of the eye that can lead to blindness) and heart disease, both of which tend to develop over time.

More recently, pancreas and islet cell transplantation, have shown some success. Annually, over 1,300 people receive pancreas transplants, with over 80% displaying no diabetic symptoms and are not required to take insulin to maintain their normal blood glucose levels. Pancreas and islet cell transplantation therapies, however, are limited by the availability of donor cadavers. Furthermore, to prevent the body from rejecting the transplanted pancreas or islet cells, patients must take powerful immunosuppressive drugs for the rest of their lives. Immunosuppressive drugs, however, makes patients susceptible to a host of other diseases. Many hospitals will not perform a pancreas transplant unless the patient also needs a kidney transplant because the risk of infection due to immunosuppressant therapy can be a greater health threat than the diabetes itself.

Recently, advances in cell-replacement therapy for diabetes and the shortage of transplantable islet cells have led to an interest in generating a new source of renewable insulin-producing cells, which could be used for transplantation. The progress over the last several years clearly indicates that the stem cell technology may provide the basis for β-cell replacement therapy. Currently, several approaches are being explored to generate insulin-producing cells in vitro, either by genetic engineering of β-cells or utilizing a wide variety of stem or progenitor cells lines. The current stem cell research efforts have been divided between embryonic and tissue specific adult stem cells as potential therapeutic progenitor cells. Recent experiments with embryonic stem (ES) cells have demonstrated that these highly proliferative, pluripotent cells can differentiate into pancreatic-like β-cells. The major problem with ES cells is their pluripotency and the risk that these cells, once transplanted, could induce the formation of tumors. Given that, adult tissue specific stem cells and their progeny have become extremely attractive as a potential cell therapeutic.

Tissue specific stem cells have two distinct advantages over ES cells; first, these cells can be isolated from a mom manageable source such as bone marrow, peripheral blood or other tissues and secondly, they exhibit the capacity to differentiate into a variety of cell lineages under controlled conditions. Stem cell based therapies in which pancreatic insulin-producing cells are generated through controlled differentiation would be beneficial for providing a novel treatment for diabetes. Thus, needs exist in the art to develop a renewable source of human stem cells that can be differentiated from adult stem cells. These adult stem cells should be relatively accessible in order to develop cell types from suitable populations that can be developed in a therapeutic method for production of human pancreatic islet cells. The use of autologous stem cells will provide a therapy for the treatment of diseases and amelioration of symptoms of diabetes.

### SUMMARY

Provided herein are methods for generating a pancreatic islet-like cell, or monocyte-derived islet cell (MDI). A stem cell may be induced to differentiate into a MDI by contacting the stem cell with at least one differentiation factor. The differentiation factor may be anti-CD40 antibody, epidermal growth factor (EGF), exendin-4, hepatocyte growth factor (HGF), insulin-like growth factor-1 (IGF1), insulin-like growth factor-2 (IGF2), LPS, nicotinamide, or combinations thereof. The MDI may express any of the following genes: insulin, IGF2, somatostatin, ngn3, PDX1, islet1, glucose transporter 2 (Glut2), and combinations thereof. The stem cell may express CD117, c-peptide, DPPA5, HES-1, OCT-4, SSEA4, or combinations thereof. The stem cell may be an adult stem cell. The stem cells may be derived from a peripheral blood monocyte. The stern cell may be in a serum-free medium, which may be Megacell DMEM/F12. The stem cell may be isolated from a patient having type 1 or type 2 diabetes.

The MDI may be an α-, γ-, or δ-like cell. A plurality of MDIs may be α-, β-, γ-, or δ-like cells, or a combination thereof. The MDI may secrete insulin in response to an insulin agonist, such as glucose, tolbutamine, and combinations thereof. The MDI may be used to treat a pancreatic-related disorder, such as type 1 diabetes, type 2 diabetes, hyperglycemia, hyperlipidernia, obesity, Metabolic Syndrome, and hypertension.

Also provided herein is a method of treating diabetes, which may comprise administering to a patient in need thereof a MDI.

### BRIEF DESCRIPTION OF THE DRAWINGS

FIG. 1 depicts photomicrographs of monocyte-derived stem cells cultured under different conditions. Panels A and D show two different preparations of cells maintained in de-differentiation medium. Panels B and C show different magnifications of a preparation of cells after 18 hours in pancreatic differentiation medium. Panels E and F show different magnifications of another preparation of cells after 18 hours in pancreatic differentiation medium.

FIG. 2 depicts photomicrographs of clusters of islet-like cells after 2-3 days in pancreatic differentiation medium. Lower right-hand panel presents control cells maintained in de-differentiation medium.

FIG. 3 depicts a graph illustrating the expression of pancreatic genes during days 1-12 of pancreatic differentiation. Gene expression was analyzed by real-time PCR. Presented are the expression profiles of cells grown in de-differentiation medium (de-diff) or pancreatic differentiation medium (Pan diff) in the presence of low or high concentrations of glucose.

FIG. 4 depicts a graph illustrating the expression of pancreatic genes during days 1-12 of pancreatic differentiation. Gene expression was analyzed by real-time PCR. Presented are the expression profiles of cells grown in de-differentiation medium (de-diff) or pancreatic differentiation medium (Pan diff) in the presence of low or high concentrations of glucose.

FIG. 5 depicts a graph illustrating the secretion of insulin by MDI clusters. Presented is the amount of insulin in cultures of cells grown in de-differentiation medium (de-diff) or pancreatic differentiation medium (Pan cliff) in the presence of low or high concentrations of (glucose.

FIG. 6 depicts a graph illustrating the secretion of c-peptide by MDI clusters. Presented is the amount of c-peptide in cultures of cells grown in de-differentiation medium (de-diff) or pancreatic differentiation medium (Pan cliff) in the presence of low or high concentration of glucose.

FIG. 7 depicts a graph illustrating the secretion of insulin by MDI clusters in response to glucose and tolbutamide. Presented is the amount of insulin in cultures of pancreatic cells exposed to increasing concentrations of glucose with or without tolbutamide,

FIG. 8 depicts a graph illustrating the percentage of monocyte-derived stem cells (MDSCs) or monocyte-derived islet cells (MDIs) expressing Ki-67 protein, a marker of cell proliferation, in response to de-differentiation medium and glucose over a 17-day period.

FIG. 9 depicts a graph illustrating the number of MDIs generated from MDSCs exposed to pancreatic medium and either low (5 mM) or high (25 mM) levels of glucose.

FIG. 10 depicts a graph illustrating MDI cluster size (in μm) in response to low (squares) or high levels (diamonds) of glucose over a 20-day period.

FIG. 11 depicts photomicrographs of the expression of the β-cell marker insulin in small (A,C) and large (B) MDI clusters after 21 days in culture. Expression of the a-cell marker glucagon was also detected in MDI cultures processed by cytospin (D). (A) and (B) are shown at 200× magnification. (C) and (D) are shown at 400× magnification.

FIG. 12 depicts photomicrographs of the expression of the β-cell markers C-peptide (A) and Pdx1 (B) in MDI clusters after 21 days in culture. (A) and (B) are shown at 600× and 200× magnification, respectively.

FIG. 13 depicts photomicrographs of MDSCs generated from peripheral blood monocyte cells (PBMCs) of human subjects with type 1 diabetes. PBMCs were incubated for 6 days in de-differentiation medium to form MDSCs. (A) MDIs formed from MDSCs treated with de-differentiation medium, 5 mM glucose (i.e., pancreatic medium), after 8 days in culture, (B) MDIs aggregated into free floating clusters after 6 days in pancreatic medium, (C) and (D) MDI clusters with increased number in size after 6 days in pancreatic medium with high glucose levels (25 mM). Scale bars in (A), (B), and (C) and (D) indicate 20 μm, 70 μm, and 110 μm, respectively.

FIG. 14 depicts photomicrographs of β-cell and a-cell marker expression in MDIs derived from human subjects with type 1 (A-C) and type 2 (D-E)diabetes. (A) and (D) show expression of the β-cell marker C-peptide, (B) and (E) show expression of the α-cell marker glucagon, and (C) and (F) show expression of the β-cell marker Pdx-1. Scale bars represent 30 μm.

FIG. 15 depicts a graph illustrating insulin levels (ng/mL) in plasma from subjects' blood (“plasma”), and in supernatant collected during MDI growth (d15-d40) from MDI derived from subjects with diabetes, as measured by ELISA and Luminex.

FIG. 16 depicts a graph illustrating blood glucose levels (mg/dL) in NOD/SCID mice that were wildtype (grey diamonds), streptozotocin (STZ)-treated (squares), STZ-treated followed by injection with MDSCs (triangles), STZ-treated followed by injection with d15 MDIs (circles), or STZ-treated followed by injection with d23 MDIs (black diamonds).

FIG. 17 depicts a graph illustrating body weight (g) of NOD/SCID mice that were wildtype, streptozotocin-treated, STZ-treated followed by injection with MDSCs, or STZ-treated followed by injection with d15 MDIs.

FIG. 18 depicts photomicrographs of insulin (A, B) and glucagon (C, D) expression in MDIs injected under the kidney capsule of STZ-treated NOD/SCID mice injected with d15 MDIs. (B) and (D) are higher magnifications of the kidney capsule areas shown in (A) and (C), respectively.

### DETAILED DESCRIPTION

### 1. Method of Generating MDIs

Provided herein is a method for generating MDIs. The cells may be composed of pancreatic α-, β-, γ-, or δ-like cells or a group thereof. The MDI may be generated by contacting an isolated monocyte-derived stem cell with a differentiation factor. The differentiation factor may be anti-CD40 antibody, EGF, exendin-4, HGF, IGF1, IGF2, lipopolysaccharide (LPS), nicotinamide, or combinations thereof. Exposure to the differentiation factor may cause the stem cell to differentiate into a MDI. The MDIs may be generated or grown in a serum-free media, such as Megacell DMEM/F12. A serum-free medium may be without a serum, such as FBS (fetal bovine serum) or Human AB serum.

The MDI may express β-cell markers such as insulin, c-peptide, islet1, IGF2, ngn3, PDX1, Glut2; or δ-cell markers such as somatostatin, or α-cell markers including but not limited to glucagon. The MDI may secrete insulin in response to glucose, tolbutamine or other insulin agonists or antagonists of insulin and combinations thereof.

a. Stein Cell

The stem cell may be de-differentiated from a monocyte. The monocyte may be derived from human peripheral blood. The monocyte may be de-differentiated by contact with leukocyte inhibitory factor (LIF), macrophage colony-stimulating factor (M-CSF), or a combination thereof. The de-differentiated stem cell may express stem cell-specific markers, such as CD 117, DPPA5, HES-1, OCT-4, SSEA4, or combinations thereof. In addition, the pancreatic islet-like cluster may secrete a pancreatic factor or hormone including, but not limited to, insulin, c-peptide, glucagon and combinations thereof.

b. Differentiation

The stem cell may be differentiated into a MDI by contact with a differentiation factor or more than one factor in combination. The differentiation factor may be CD40 antibody, EGF, exendin-4, HGF, IGF1, IGF2, LPS, nicotinamide, and combinations thereof. The concentration of CD40 antibody may range from 10 ng/ml to 2 μg/ml. The concentration of EGF may range from 10 ng/ml to 50 ng/ml. The concentration of exendin-4 may range from 10 mM to 40 mM. The concentration of HGF may range from 10 ng/ml to 50 ng/ml. The concentration of IGF1 may range from 10 ng/ml to 50 ng/ml. The concentration of IGF2 may range from 10 ng/ml to 50 ng/ml. The concentration of LPS may range from 10 ng/ml to 100 ng/ml. The concentration of nicotinamide may range from 5 mM to 20 mM. The differentiation factor may be presented to the cells in the presence of culture medium. The culture medium may be LDMEM (low glucose DMEM), HDMEM (high glucose DMEM), DMEM/F12, or Megacell DMEM/F12. The culture medium may be supplemented with serum or serum proteins. Alternatively, the cells may be grown in culture medium without added serum or serum proteins. The differentiation medium may comprise glucose, which may be at a concentration of 2-15 mg/dL or 5-8 mg/dL. The differentiation medium may be changed every three days for optimal differentiation.

Differentiation may be monitored by a variety of methods known in the art. Changes in a parameter between a stem cell and a differentiation factor-treated cell may indicate that the treated cell has differentiated. Microscopy may be used to directly monitor morphology of the cells during differentiation. As an example, the differentiating pancreatic cells may form into aggregates or clusters of cells. The aggregates/clusters may contain as few as 10 cells or as many as several hundred cells. The aggregated cells may be grown in suspension or as attached cells in the pancreatic cultures.

Changes in gene expression may also indicate pancreatic differentiation. Increased expression of pancreatic-specific genes may be monitored at the level of protein by staining with antibodies. Antibodies against insulin, Glut2, Igf2, islet amyloid polypeptide (IAPP), glucagon, neurogenin 3 (ngn3), pancreatic and duodenal homeobox 1 (PDX1), somatostatin, c-peptide, and islet-1 may be used. Cells may be fixed and immunostained using methods well known in the art. For example, a primary antibody may be labeled with a fluorophore or chromophore for direct detection. Alternatively, a primary antibody may be detected with a secondary antibody that is labeled with a fluorophore, or chromophore, or is linked to an enzyme. The fluorophore may be fluorescein, FITC, rhodamine, Texas Red, Cy-3, Cy-5, Cy-5.5. Alexa⁴⁸⁸, Alexa⁵⁹⁴, QuantumDot⁵²⁵, QuantumDot⁵⁶⁵, or QuantumDot⁶⁵³. The enzyme linked to the secondary antibody may be HRP, β-galactosidase, or luciferase. The labeled cell may be examined under a light microscope, a fluorescence microscope, or a confocal microscope. The fluorescence or absorbance of the cell or cell medium may be measured in a fluorometer or spectrophotomer.

Changes in gene expression may also be monitored at the level of messenger RNA (mRNA) using RT-PCR or quantitative real time PCR. RNA may be isolated from cells using methods known in the art, and the desired gene product may be amplified using PCR conditions and parameters well known in the art. Gene products that may be amplified include insulin, insulin-2, Glut2, Igf2, LAPP, glucagon, ngn3, PDX1, somatostatin, ipf1, and islet-1. Changes in the relative levels of gene expression may be determined using standard methods. The expression of α-, β-, γ-, and δ-cell specific markers may show that the MDIs, aggregates or clusters of cells derived from monocyte-derived stem cells (MDSCs) are composed of all four distinct types and three major types of pancreatic cells.

The formation of functional monocyte-derived islets (MDIs) may be determined by monitoring the synthesis and secretion of factors such as insulin and c-peptide during the differentiation of MDSC-derived MDIs. Contact with high levels of glucose may stimulate the MDIs to secrete insulin or c-peptide. Contact with tolbutamide or other insulin agonists may stimulate the MDIs to secrete increased levels of insulin. The levels of insulin or c-peptide may be measured in the culture medium of the different cells the using an ELISA protocol. Other methods known in the art may be used to monitor the secretion of insulin or c-peptide by the differentiated cells.

c. Proliferation

The MDI may be induced to proliferate by contacting it with differentiation medium comprising glucose, which may be at a concentration of 5-40 mg/dL, 10-25 mg/dL, or 18-25 mg/dL. The proliferation may be monitored by staining the MDI with propidium iodide or Ki-67, which may be followed by flow cytometry.

### 2. Methods of Using the MDI

The MDI may be used to replenish a cell population that has been reduced or eradicated by a disease or disorder, as a treatment for such a disease or disorder, or to replace damaged or missing cells or tissue(s). The MDI may be given autologously or to a allogenically compatible subject.

Diabetes mellitus is an example of a disease state associated with an insufficiency or effective absence of certain types of cells in the body. In this disease, pancreatic islet β-cells are missing or deficient or defective. The condition can be treated, or at least one of its symptoms ameliorated, by insertion of MDIs. The MDIs may be derived from MDSC isolated from a patient that is healthy, or who may have type 1 or type 2 diabetes. Both type 1 diabetes mellitus (juvenile-onset diabetes or insulin-dependent diabetes mellitus) and type 2 diabetes mellitus (adult-onset diabetes) may be treated with MDIs. Other disorders that may be treated with MDIs include hyperglycemia, hyperlipidemia, obesity, Metabolic Syndrome, and hypertension.

MDIs may be inserted into the body by implantation, transplantation, or injection of cells. The cells may be introduced as single cells or clusters of cells. Methods of transplanting pancreatic cells are well known in the art. See for example, U.S. Pat. Nos. (4,997,443 and 4,902,295) that describe a transplantable artificial tissue matrix structure containing viable cells, preferably pancreatic islet cells, suitable for insertion into a human. Moreover, since MDIs may be derived from peripheral blood monocytes of the same individual who will later receive the cell transplantation, the use of immunosuppressive agents may not be necessary.

### 3. Compositions of MDIs

Also provided herein are compositions comprising the MDIs. The compositions may include a single cell, an aggregate of cells. or a tissue-like cluster of cells. The composition may comprise 10-10,000, 10-1000, or 10-1000 MDIs. The composition may also comprise 5-60% α-cells, 30-95% β-cells, 1-30% δ-cells. 0-5% γ-cells, or combinations thereof.

As various changes could be made in the above compounds, methods, and products without departing from the scope of the invention, it is intended that all matter contained in the above description and in the examples given below, shall be interpreted as illustrative and not in a limiting sense,

The following examples illustrate, but do not limit, the invention.

### EXAMPLE 1

### Differentiation of MDIs

Isolated peripheral blood monocytes were plated in a 2:1 mixture of Megacell DMEM/F12 medium (Cat. No. M4192, Sigma-Aldrich) and AIM V medium (Invitrogen) and cultured overnight at 37° C. and 5% CO₂. The culture medium was supplemented with 4 mM L-glutamine and penicillin-streptomyocin. The cells were plated on FALCON vacuum-gas plasma treated plates. After 24 hours, the culture medium was removed and the cells were gently washed three times with 1× HBSS containing 2 mM EDTA. De-differentiation medium, which was Megacell DMEM/F12 or LDMEM (low glucose DMEM) or HDMEM (high glucose DMEM) containing 10 ng/ml leukocyte inhibitory factor (LIF; Cat. No. LIF1010, Chemicon) and 25 ng/ml macrophage colony-stimulating factor (M-CSF: Cat. No. GF053, Chemicon), was added. After three days, the medium was removed and replaced with fresh de-differentiation medium. After 6 days in culture the cells had de-differentiated into monocyte-derived stem cells (MDSCs).

MDSCs were washed two times with 1× HBSS. Pancreatic differentiation medium was added to the cells and they were cultured. Pancreatic differentiation medium comprised Megacell DMEM/F12 (or LDMEM or HDMEM) supplemented with L-glutamine, penicillin, and streptomyocin, as well as 1 μg/ml CD40 antibody (R&D Systems; catalog number MAB6321, clone 82111), 100 ng/ml LPS (Chemicon; catalog number LPS25), 1× ITS, 10 mM nicotinaminde, 1% N2 supplement, 25 ng/ml EGF (Chemicon; catalog number GF001), 20 ng/ml HGF (Chemicon; catalog number GF116), 25 ng/ml IGF1 (Chemicon; catalog number GF006), 25 ng/ml IGF2 (Chemicon; catalog number GF007), and 20 mM Exendin-4 (Sigma-Aldrich; catalog number E7144).

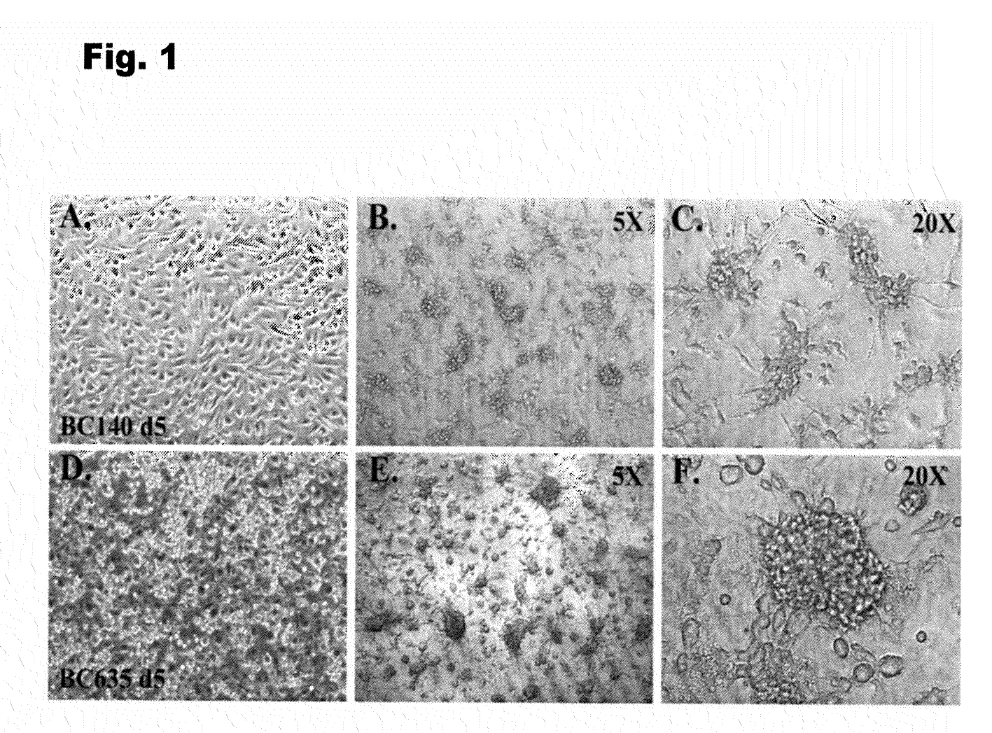

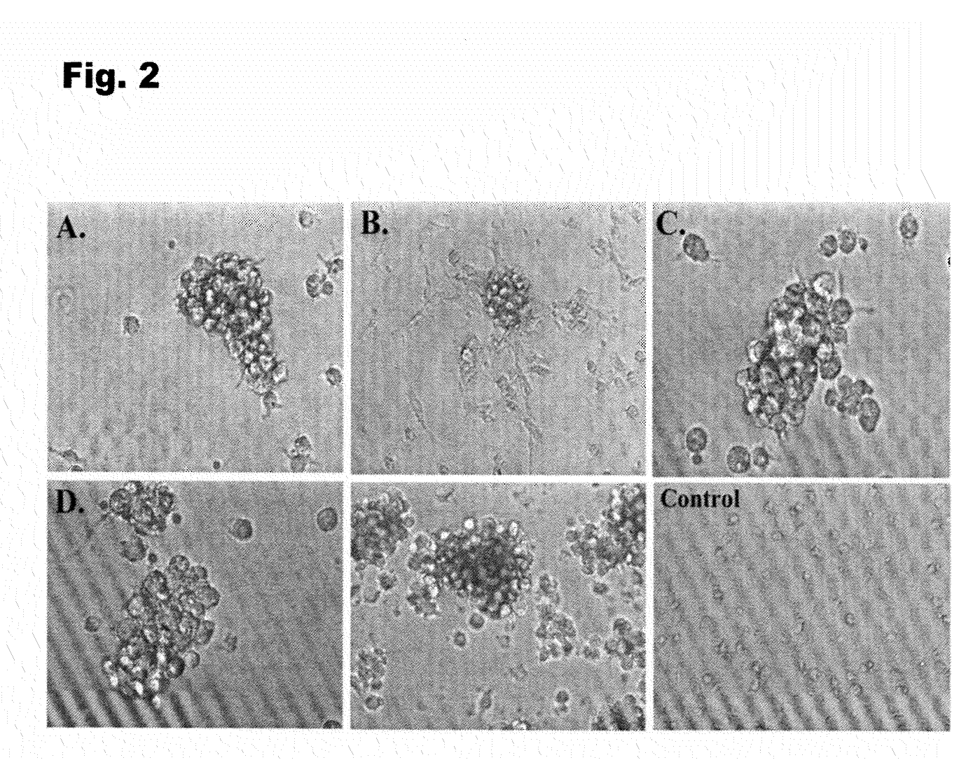

Aggregates of cells were observed after 18 hours in pancreatic differentiation medium (FIG. 1). The number and size of aggregates increased over the next several days (FIG. 2). The pancreatic islet aggregates or clusters were composed of a variety of different sized cells that ranged in total number from approximately 10 cells to hundreds of cells per aggregate or cluster. The number and size of the aggregates appeared to depend upon the initial cell density of the MDSCs. Typically, cultures that were initially seeded at higher density generated more and larger aggregate clusters than cultures from initially lower density cultures.

After 6 days in culture, the aggregates detached from the plates and were free floating clusters. Beginning at 4-6 days, pancreatic factors or hormones such as insulin, c-peptide and glut2 were initially detected in MDIs derived from MDSCs that were cultured under pancreatic differentiation conditions, while no pancreatic factors or hormones were detected in de-differentiated MDSC cultures. At this time, the cells were challenged with high glucose conditions. For these experiments, cells were exposed to pancreatic differentiation medium containing 25 mM glucose (normal pancreatic differentiation medium contained 5 mM glucose). The number and size of the aggregates or clusters increased in the presence of high glucose conditions. In addition, the expression of several genes was also changed (see Example 2). Cultures were shown to maintain their growth over a month by changing the pancreatic differentiation medium containing 25 mM glucose every three days.

### EXAMPLE 2

### Pancreatic Gene Expression

To monitor the differentiation of MDSCs into MDIs, the expression of pancreatic-specific genes was analyzed by real time PCR. The following cell-specific markers were examined: β-cell specific markers were Glut2, IAPP, Igf2, insulin, ngn3, and PDX1; α-cell specific marker, glucagon; and δ-cell specific marker, somatostatin. MDSCs were generated as described in Example 1. One set of MDSCs was maintained in de-differentiation medium. The second set was cultured in pancreatic differentiation medium for six days and then challenged with high glucose conditions.

For each time point, cells were collected (1×10⁵ to 3×10⁶ cells/well) and RNA was isolated using Qiagen Rneasy Kit (Cat. No. 74103) following the manufacturer's instructions. First strand cDNA was synthesized by mixing 1 ng-5 μg of RNA with 1 of 500 μg/ml of oligo(dT) (Invitrogen; catalog number 55063), 1 μl of 10 mM dNTPs (Invitrogen; catalog number 18427-013), and water to equal 12 μl. The mixture was heated to 65° C. for 5 minutes and the chilled on ice. Then 4 μl of 5× First-strand buffer, 1 μl of 0.1 M DTT (Invitrogen; catalog number 18427-013), 40 units of RNaseOUT (Invitrogen: catalog number 10777-019), and 200 units of Superscript III RNaseH⁻ RT (Invitrogen; catalog number 18080-093) were added. The tube was gently mixed and incubated at 50° C. for 60 minutes. The tube was spun and the enzymes were inactivated by heating to 70° C. for)5 minutes. The concentration of cDNA was estimated using a spectrophotometer.

For real time (quantitative) PCR, 100 ng of cDNA was mixed with 200 nM of each primer, and 0.5 volume of SYBR green qPCR SuperMix-UDG with ROX (Invitrogen; catalog number 11744). The cycling parameters were 50° C. for 2 minutes, 95° C. for minutes, followed by 40 cycles of 60° C. for 30 seconds and 95° C. for 30 seconds. Primers were designed by Primer3 software with T_{M}=60° C. See Table 1 for primer sequences and sizes. All PCR reactions were run in duplicate and averaged based on ΔCT values. To determine the relative gene expression, the ΔCT values for controls (GADPH and β-actin) were compared to pancreatic gene expression. To calculate the percent of relative expression, the following formula was used:

R.E. (relative expression )=2ⁿ−(ΔCT gene−ΔCT GAPDH)×100

TABLE 1
PCR Primers

| | | Length | SEQ |
|---|---|---|---|
| Primer Name | Sequence | (bp) | ID NO |
| glucagon-f | GATGAAGTACCCCAACCTGTTTAC | 156 | 1 |
| glucagon-r | AAGTTCTCTTTCCAATTTCACCAC | | 2 |
| C-peptide-f | TCACCTTTGAACTTCGAGATACAG | 250 | 3 |
| C-peptide-r | CCAGAAGCTTAAAAGAAAGATTGG | | 4 |
| IGF2-f | GGGCAAGTTCTTCCAATATGAC | 166 | 5 |
| IGF2-r | GTCTTGGGTGGGTAGAGCAAT | | 6 |
| Isletl-f | ACAAGCAGCCGGAGAAGAC | 221 | 7 |
| Islet 1 -r | CTGCTGGAGTTGCTTCATCAT | | 8 |
| Glut2-f | GTTCCACTGGATGACCGAAA | 187 | 9 |
| GIut2-r | TCATTCCACCAACTGCAAAG | | 10 |
| IAPP-f | TGGCACAGGTTTAAGAACGA | 195 | 11 |
| IAPP-r | GTCAGGCGGGGTCTCGAACTC | | 12 |
| Ipf I -f | AGCTTTACAAGGACCCATGC | 175 | 13 |
| 1pfl -r | CCTCGTACGGGGAGATGT | | 14 |
| Insulin (human )-f | GAGGGGTCCCTGCAGAAG | 216 | 15 |
| Insulin (human)-r | GGTTCAAGGGCTTTATTCCA | | 16 |
| Insulin-2 (human)-f | AACGAGGCTTCTTCTACACACC | 206 | 17 |
| Insulin-2 (human)-r | CTGCGTCTAGTTGCAGTAGTTCTC | | 18 |
| Somatostatin-f | AGCTGCTGTCTGAACCCAAC | 162 | 19 |
| Somatostatin-r | AGAAATTCTTGCAGCCAGCTT | | 20 |
| PDX-1-f | ATTTCCAACTTGGGGATGTTT | 217 | 21 |
| PDX-1-r | TTTAAGAAACCTGGTTGCCAGT | | 22 |
| Ngn3-f | AATCGAATGCACAACCTCAAC | 162 | 23 |
| Ngn3-r | GTACAAGCTGTGGTCCGCTAT | | 24 |
| GAPDH-f | CAAAGTTGTCATGGATGACC | 195 | 25 |
| GAPDH-r | CCATGGAGAAGGCTGGG | | 26 |
| ACTB-f | GCTTGCTGATCCACATCTGC | 219 | 27 |
| ACTB-r | TGGACATCCGCAAAGACCT | | 28 |

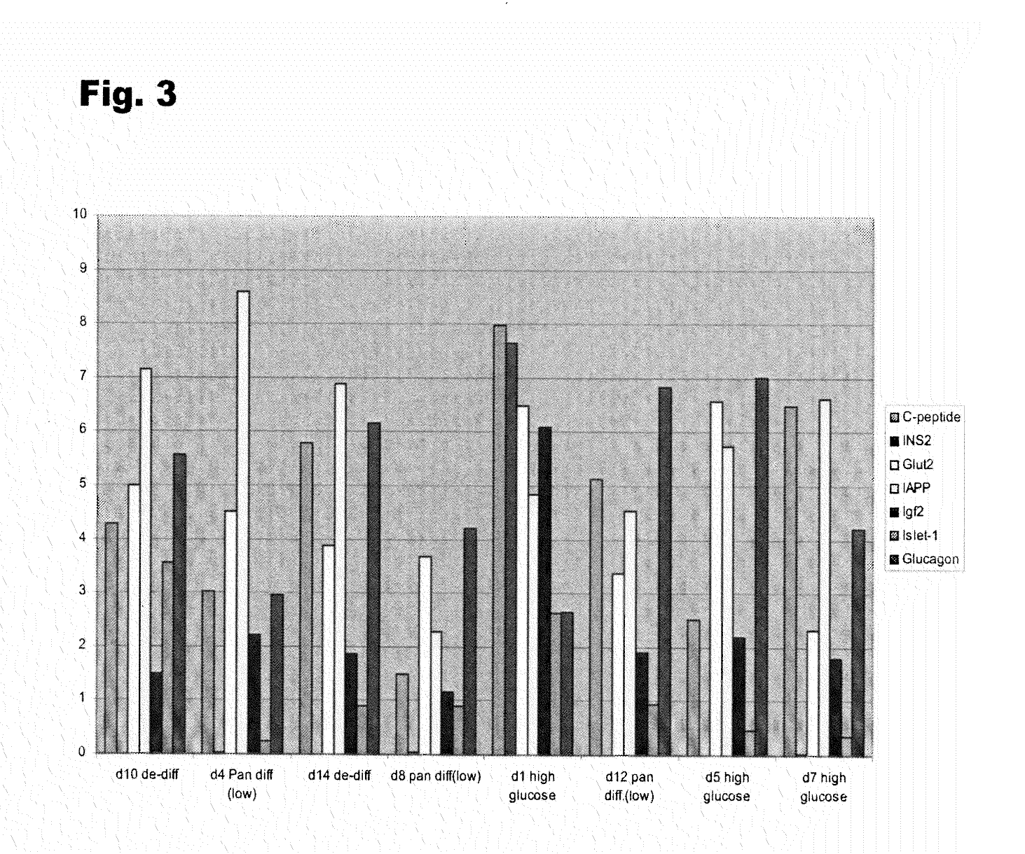

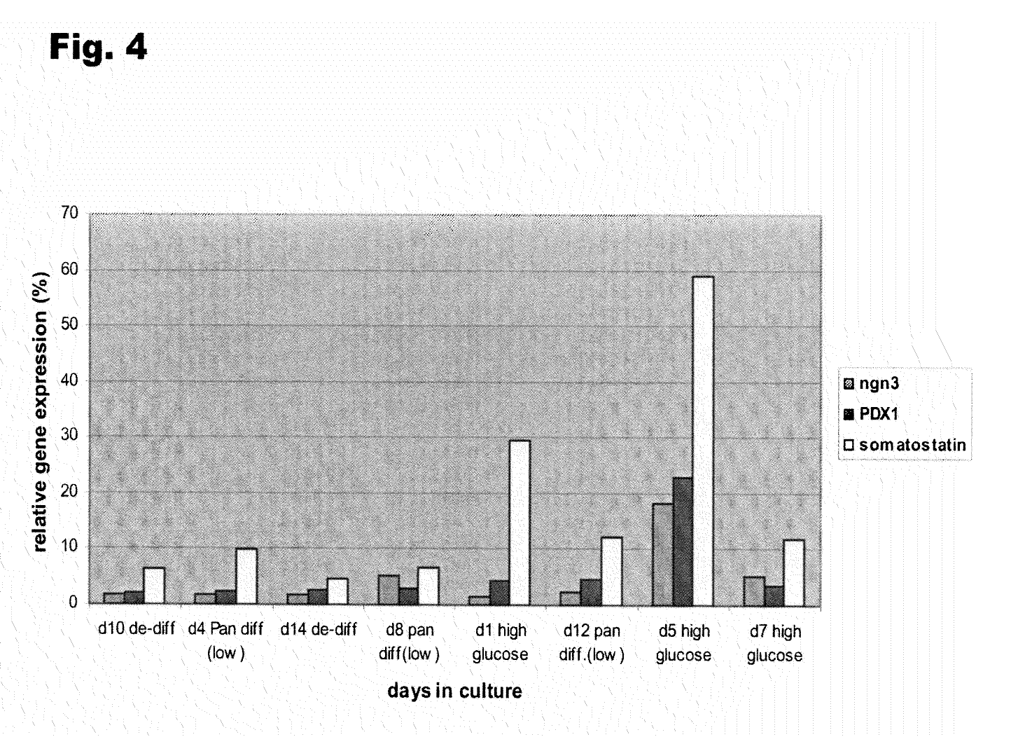

FIG. 3 presents the relative levels of expression of pancreatic-specific genes during pancreatic differentiation. There was an increased expression of insulin, c-peptide. Igf2, islet 1, and Glut2. FIG. 4 presents the percent of relative gene expression of ngn3, PDX1, and somatostatin under the different conditions.

### EXAMPLE 3

### Insulin Secretion

To assess the functionality of the differentiated MDIs, insulin secretion was measured under the different conditions using an ELISA kit (Diagnostic Systems Labs Inc; Cat. No. DSL-10-1600). For this “one-step” sandwich-type Immunoassay, standards, controls, and unknown serum samples were incubated with an HRP-labeled anti-insulin antibody in microtitration wells that had been coated with another anti-insulin antibody. After incubation and washing, the wells were incubated with the substrate tetramethylbenzidine (TMB). An acidic stopping solution was then added and the degree of enzymatic turnover of the substrate was determined by dual wavelength absorbance measurement at 450 and 620 nm. The absorbance measured was directly proportional to the concentration of insulin present. A set of insulin standards was used to plot a standard curve of absorbance versus insulin concentration from which the concentration of insulin in the unknown samples was calculated.

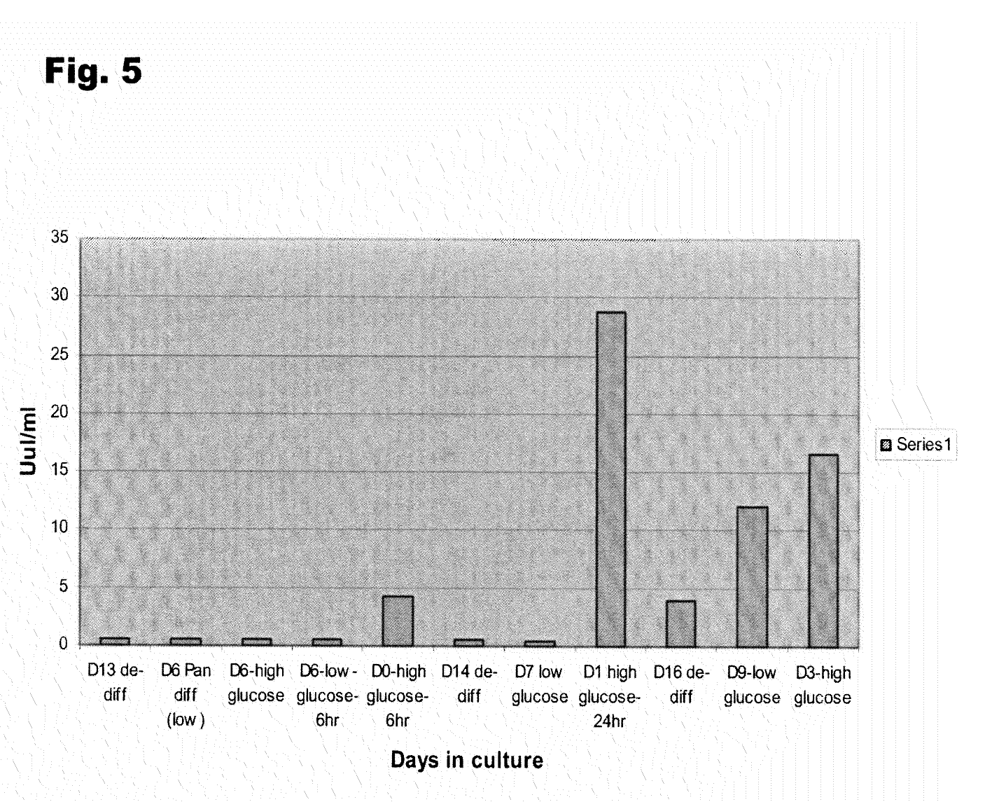

After 24 hours of high glucose challenge the pancreatic aggregates synthesized 28.8 μl U/ml of active insulin into the medium (FIG. 5). (The range for normal adult subjects after an overnight fast was 5-10 μl U/ml (basal plasma insulin) while during meal consumption ranged from 30-150 μl U/ml.) As the length of time in culture increased, greater amounts of insulin were synthesized and secreted by the aggregates of islet-like cells.

### EXAMPLE 4

### C-Peptide Secretion

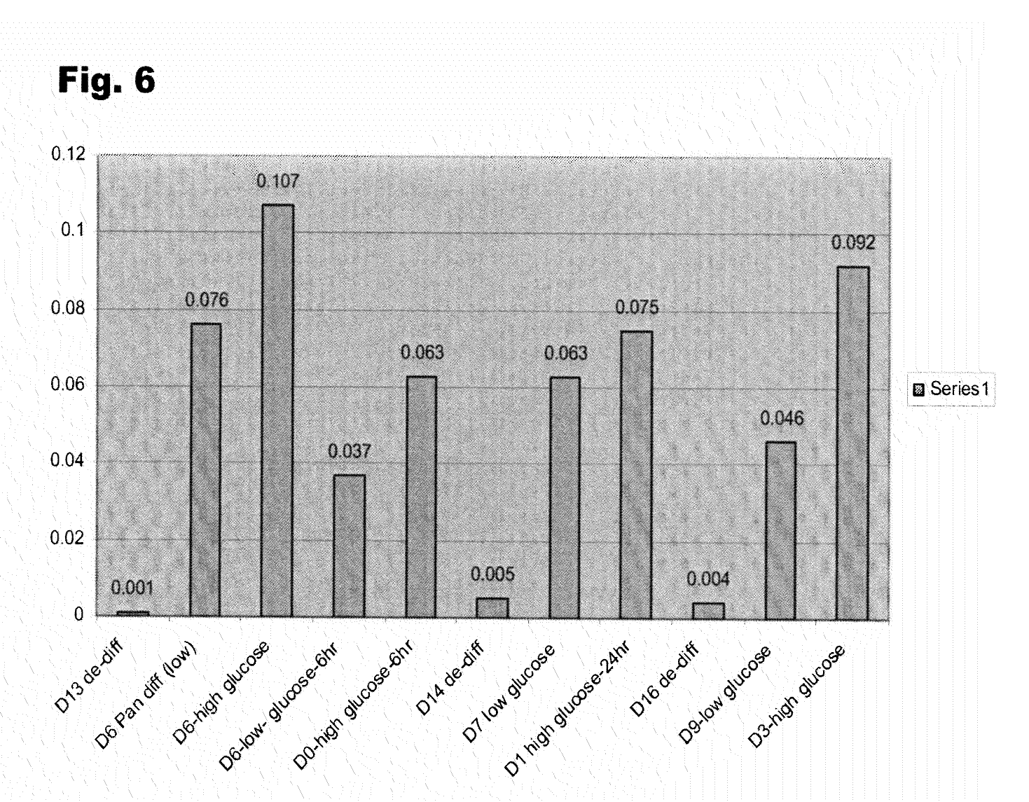

To further analyze the function of the aggregates of MDIs, an ELISA kit (Diagnostic Systems Labs Inc; Cat. No. DSL-10-7000) was utilized to measure the level of c-peptide secreted by the cells. In this assay, standards, controls and unknown serum samples were incubated with an HRP-labeled anti-c-peptide antibody in microtitration wells that had been coated with another anti-c-peptide antibody. After incubation and washing, the wells were incubated with the substrate tetramethylbenzidine (TMB), An acidic stopping solution was then added and the degree of enzymatic turnover of the substrate was determined by dual wavelength absorbance measurement at 450 and 620 nm. The absorbance measured was directly proportional to the concentration of C-peptide present. A set of c-peptide standards was used to plot a standard curve of absorbance versus c-peptide concentration from which the concentration of c-peptide in the unknown samples was calculated. FIG. 6 shows that the differentiated aggregates secreted c-peptide, whereas the de-differentiated MDSCs produced no or extremely low levels of c-peptide.

### EXAMPLE 5

### Insulin Secretion During Tolbutamide Induction

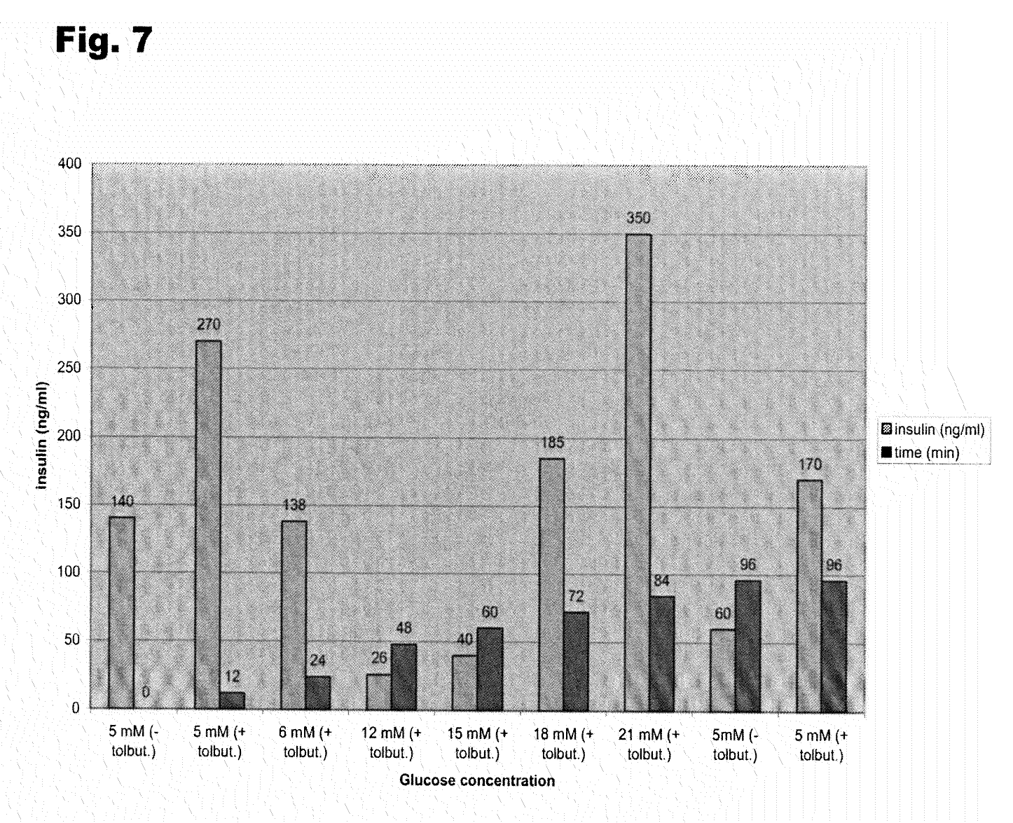

To further examine the function of these differentiated islet-like cells, the effects of tolbutamide and glucose were studied in parallel. Cells were exposed to increasing concentrations of glucose (5, 6, 7, 10, 12, 15, 18, 21 and 25 mM) in the presence or absence of 10 μM tolbutamide for periods of 12 minutes each. The secretion of insulin was analyzed using an insulin ELISA kit (see Example 3). For these experiments pancreatic clusters were collected and plated in a 24 well format with 2 ml of Krebs-ringer bicarbonate buffer containing 5 mM glucose. FIG. 7 shows that tolbutamide stimulated the secretion of insulin by these pancreatic islet clusters. Furthermore, these clusters responded to increasing glucose concentrations. These clusters generated physiologically relevant levels of insulin ranging between 140-270 ng/ml. Similar results were also observed using other insulin agonists, while the addition of insulin antagonists generally resulted in a decrease in insulin secretion.

### EXAMPLE 6

### Monocyte-Derived Islet Cells Exhibit Increased Proliferation

The following demonstrates that monocyte-derived islet cells (MDIs) exhibit increased proliferation in response to pancreatic medium and high glucose levels (25 mM). To assay the proliferation of MDSCs and MDIs the expression of Ki-67, a marker strictly associated with cell proliferation, was assayed. During interphase, this antigen can be exclusively detected within the nucleus, whereas in mitosis most of the protein is relocated to the surface of the chromosomes. The fact that the Ki-67 protein is present during all active phases of the cell cycle (G(1), S, G(2), and mitosis), but is absent from resting cells (G(0)), makes it an excellent marker for determining the so-called growth fraction of a given cell population.

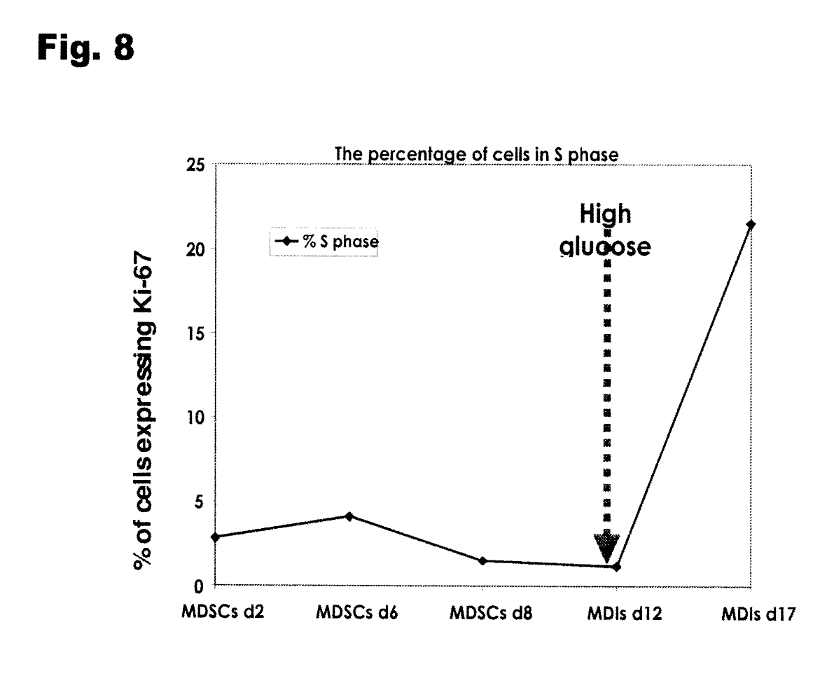

The effects of high glucose on MDI proliferation as measured by Ki-67 is shown in FIG. 8. For this analysis flow cytometry was used to count the percentage of cells that stained positive for Ki-67 in MDSCs cultures from day 2 to 12. During this experiment, MDSCs were cultured in de-differentiation medium that contained M-CSF and LIF for 6 days. After 6 days MDSCs were transferred to pancreatic medium containing low glucose (5 mM). After an additional 6 days, cultures were transferred to pancreatic medium containing high glucose (25 mM). A relatively low level of proliferation, which increased until day 6, was observed. During these first 6 days, the MDSCs underwent a period of differentiation and typically exhibited a low level of proliferation. Once treated with pancreatic medium (day 7-12), proliferation was extremely low. During this period, MDSCs exhibited several morphological changes and transitioned from a fibroblast state into a more neural appearance. In addition, the cells formed into aggregates, and eventually into free floating clusters. However, after adding a high amount of glucose at day 12, the MDIs exhibited a dramatic increase in overall cell proliferation.

This effect is further illustrated in Table 2 below, which shows the percentage of cells in S. G0/G1, and, G2/M phases at days 2, 6, 8, 12, and 17 as measured by propidium iodide (PPI) levels in flow cytometry analysis. Higher rates of proliferation were indicated by the higher percentage of cells in S phase.

TABLE 2
Proliferation of MDSCs and MDIs as measured by PPI

| | % S phase | G0/G1 | G2/M |
|---|---|---|---|
| MDSCs d2 | 2.83 | 94.69 | 1.77 |
| MDSCs d6 | 4.13 | 77.03 | 8.68 |
| MDSCs d8 | 1.55 | 96.49 | 1.03 |
| MDIs d12 | 1.2 | 96.75 | 1.28 |
| MDIs d17 | 21.5 | 66.96 | 4.91 |

The above results indicate that pancreatic medium with high levels of glucose increases MDI proliferation.

### EXAMPLE 7

### High Glucose Levels Increase the Number of Monocyte-Derived Islet Cell Aggregates

The following demonstrates that high glucose levels increase the number of MDI. aggregates. MDSCs were cultured in serum free conditions in DMEM/F12 medium for 6 days, and then cultured in pancreatic medium containing 5 mM glucose. Pancreatic aggregates formed into small free floating clusters after 3 days in pancreatic medium. In low glucose conditions (5 mM), the cultures generated approximately 200 clusters per well in a 6 well plate (Falcon). However, when MDIs were cultured in high glucose (25 mM), approximately 600 clusters were generated per well in a 6 well plate. For these studies 20×10⁶ PBMCs per well were plated.

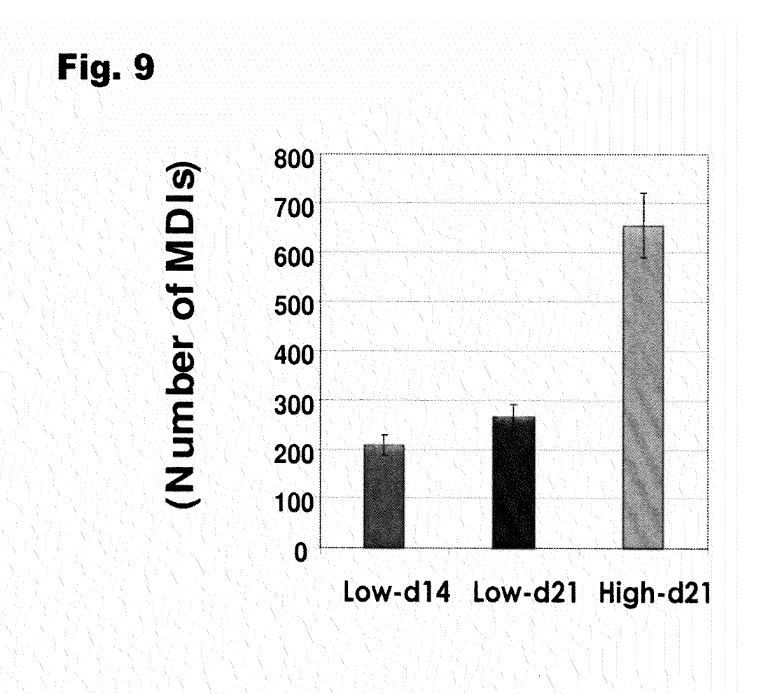

FIG. 9 shows the results of these experiments, which indicate that the number of MDIs generated in culture depended on glucose levels. The number of MDIs grown in a 6-well dish format were counted. Several different MDIs cultured were counted at both low and high glucose concentrations in pancreatic differentiation medium. An increase in the total number of clusters after treatment with high glucose conditions (at day 21), but not after treatment with low glucose, was observed. The above results indicate that pancreatic medium with high levels of glucose increase the number of MDI aggregates.

### EXAMPLE 8

### High Glucose Levels Increase Monocyte-Derived Islet Cell Cluster Size

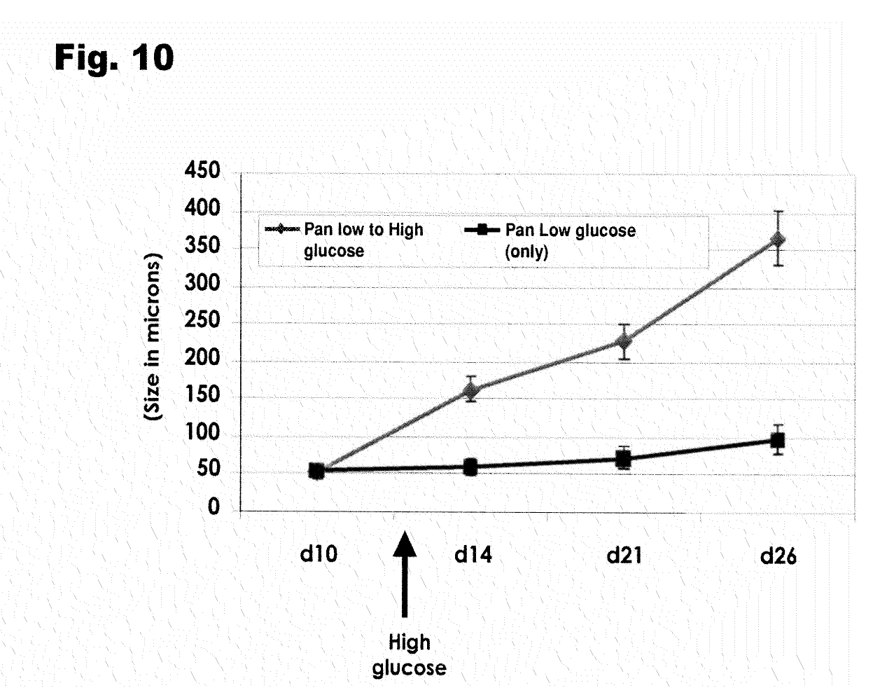

The following demonstrates that high glucose levels increase MDI cluster size. MDSCs were cultured in serum free conditions DMEM/F12 medium containing LIF and M-CSF for 6 days for the initial de-differentiation. After 6 days, MDSCs were treated with pancreatic medium containing 5 mM glucose. During this period, pancreatic aggregate formation was observed. Continued treatment of cells with pancreatic medium with low glucose eventually produced free floating clusters. After 6 days in low glucose pancreatic medium, MDIs were treated with low- or high-glucose (5 mM or 25 mM, respectively). Under these conditions increases in both size and number of MDIs in culture were observed. The results of these experiments is shown in FIG. 10, which indicates the diameter of MDIs clusters at various stages (d10, d14, d21 and (d26).

Table 3 shows the size of the MDIs in microns using a Leica DMire2 microscope with 5.1 scope imaging software. Multiple samples were measured from 6 different MDI cultures and the mean value of the size was calculated and plotted.

TABLE 3
MDI Size
Measurement of MDIs

| SAMPLE | d10 | d14 | d21 | d26 |
|---|---|---|---|---|
| BC37-1 | 47 | 170 | 190 | 278 |
| BC37-2 | 76 | 101 | 182 | 324 |
| BC21-1 | 72 | 264 | 244 | 308 |
| BC21-2 | 80 | 200 | 173 | 319 |
| BC27-1 | 77 | 124 | 161 | 332 |
| BC27-2 | 57 | 170 | 294 | 264 |
| BC27-3 | 67 | 163 | 231 | 316 |
| BC44-1 | 76 | 195 | 296 | 399 |
| BC44-2 | 49 | 137 | 320 | 403 |
| BC26-1 | 51 | | 221 | 320 |
| BC26-2 | 49 | | 234 | 560 |
| BC26-3 | 53 | | 212 | 404 |
| BC23-1 | | | | 386 |
| BC23-2 | | | | 347 |
| BC23-4 | | | | 302 |
| BC23-5 | | | | 525 |
| mean size | 62.0 | 170.0 | 225.8 | 328.0 |
| std dev | 12.9 | 47.9 | 51.5 | 82.7 |

The above results indicate that high levels of glucose in pancreatic medium increase MDI size and number.

### EXAMPLE 9

### Monocyte-Derived Islet Cells Exhibit Increased Insulin and Glucagon Expression

The following demonstrates that MDIs derived from MDSCs using pancreatic medium with high glucose levels express endocrine-specific markers in association with increased rates of proliferation. For these experiments, expression of endocrine-specific markers was examined by immunofluorecence using antibodies specific for β-cells, including insulin, c-peptide, and Pdx1, and for α-cells (glucagon). The expression profiles of these factors in MDIs were observed at various stages.

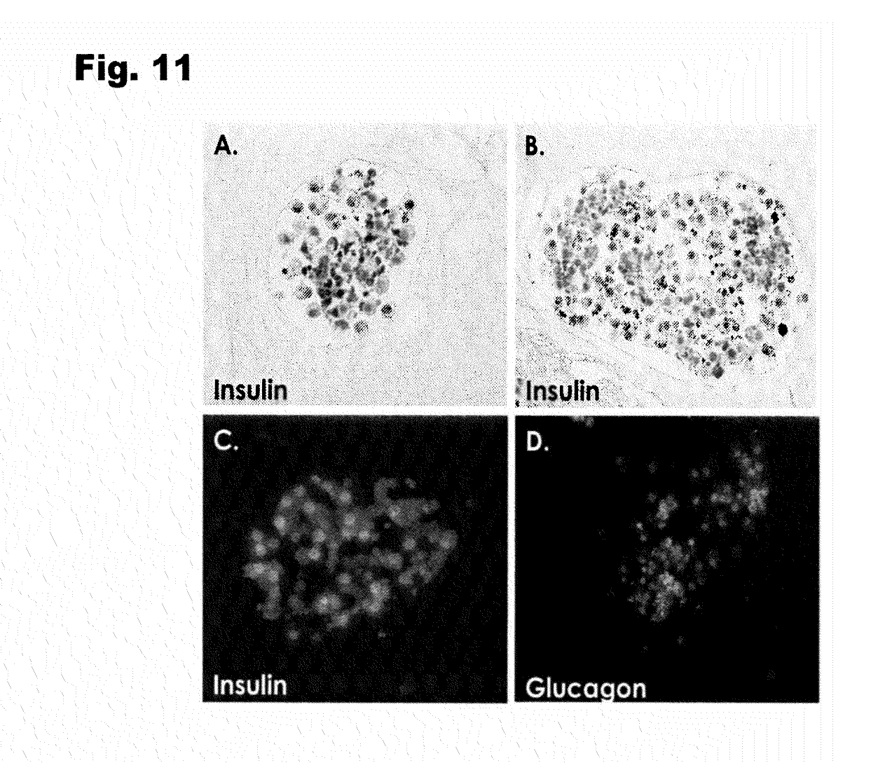

FIG. 11 shows insulin and glucagon expression in day 21 MDIs. Insulin expression was detected in day 21 MDI clusters (A-C). Approximately 70% of the cells within the small cluster (A) and larger clusters (B) expressed insulin. Using, immunofluorescence on a different MDI culture, insulin was detected in greater than 70% of the cells (C). MDIs were also stained with antibodies against glucagon after processing by cytosopin (D). Insulin- and glucagon-positive cells within the MDI cultures indicated the presence of β-cells and a-cells, respectively.

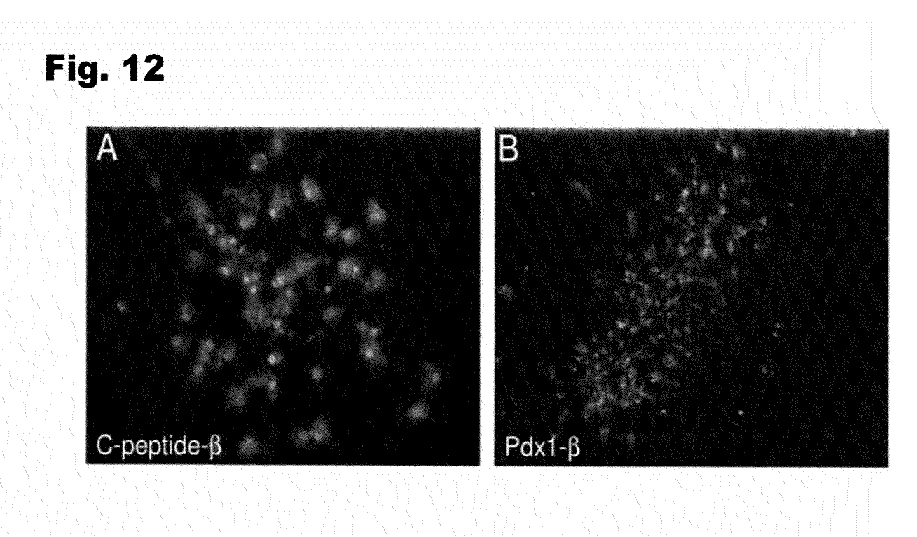

FIG. 12 shows c-peptide (A) and Pdx-1 (B) expression in day 21 MDIs, indicating the presence of β-cells. MDIs were stained with c-peptide and Pdx1 after 21 days in culture and cytospins were performed.

The results above demonstrate that MDIs express endocrine specific markers and are composed of the major pancreatic cell types (α, β and δ). Real time PCR showed that ngn3, a known marker for the pancreatic progenitors known as the γ-cells or PP cells, was expressed. The composition of the MDIs was approximately >60% β-cells, 10-25% α-cells, and 1-5% δ-cells. MDI exhibited a similar cellular composition to that observed in human pancreatic islets.

Furthermore, MDIs have an increased rate of proliferation when cultured in high glucose conditions. This increased proliferation correlates with an increased expression of ngn3, pdx1 and somatostatin biomarkers for the formation of new islet progenitors within the MDIs cultures.

### EXAMPLE 10

### Monocyte-Derived Islet Cells can be Generated from Monocyte-Derived Stem Cells of Diabetic Subjects

The following demonstrates that MDIs can be derived from MDSCs of diabetic subjects. To test the ability to generate both MDSCs and MDIs from both type 1 and 2 diabetic subjects, peripheral blood monocytes (PBMCs) were isolated from subjects with diabetes and MDSCs were produced using de-differentiation medium. To determine if functional MDIs can be generated from MDSCs derived from subjects with diabetes, their MDSCs were cultured under pancreatic differentiation conditions.

PBMCs were isolated from 14 subjects with diabetes. These subjects were diagnosed with insulin-dependent type 1 or type 2 diabetes. Multiple blood draws were performed on each of these subjects, and each draw was separated by at least 2 weeks. This provided duplicate samples to ensure reproducibility.

MDSCs were isolated and generated using methods as described above for deriving pancreatic islets, and were monitored for up to 30 days in culture. To monitor c-peptide c-peptide ELISA (DSL) and Western blot analysis were performed. Immunohistochemical and PCR analyses were performed on samples to examine the expression of several pancreatic and proliferative markers during the course of the islet formation. Luminex was used to examine the levels of insulin, c-peptide and glucagon in each subjects' plasma.

The results of generating MDSCs and MDIs from subjects with diabetes is summarized in Table 4 below.

TABLE 4

| Subjects | type 1/2 | MDSCs | MDIs |
|---|---|---|---|
| 1 | type 1 | yes | (+) |
| 2 | type 1 | yes | (+) |
| 3 | type 1 | yes | (++) |
| 5 | type 2 | yes | (++) |
| 6 | type 2 | yes | (++) |
| 7 | type 2 | yes | (++) |
| 8 | type 2 | yes | (++) |
| 9 | type 2 | yes | (++) |
| 10 | type 2 | yes | (++) |
| 11 | type 1 | yes | (++) |
| 12 | type 1 | yes | (++) |
| 13 | type 1 | yes | (+) |
| 14 | type 1 | yes | (+) |

(+) indicates the formation of smaller MDIs, typically between 50 to 100 cells per cluster; and
(++) indicates the formation of larger MDIs, typically >200 cells per cluster after treatment with high glucose conditions.

Additionally, levels of insulin, glucagon, and glp-1 in plasma collected from diabetic subjects were measure by performing a Luminex assay (Linco) according to the manufacturer's protocol. This provided baseline levels for these specific hormones. 25 μL of plasma was used for each assay and all samples were run in duplicate to provide more accurate and reliable data.

TABLE 5

| | | C-peptide | Glp-1 | Glucagon) | Insulin |
|---|---|---|---|---|---|
| | | (pM) | (pM) | (pM) | (pM) |
| Standards | 6.2 pM | 6.08 | 5.47 | 7.79 | 5.08 |
| | 6.2 pM | 6.31 | 5.47 | 4.84 | 6.73 |
| | 18.5 pM | 18.6 | 18.7 | 18.7 | 19.8 |
| | 18.5 pM | 18.5 | 19 | 17.6 | 18.1 |
| | 55.6 pM | 55.6 | 56.5 | 55.1 | 56.3 |
| | 55.6 pM | 55.6 | 52.5 | 59 | 53.6 |
| | 166.7 pM | 172 | 189 | 179 | 173 |
| | 166.7 pM | 161 | 160 | 141 | 164 |
| | 500 pM | 514 | 535 | 929 | 531 |
| | 500 pM | 503 | 441 | 561 | 465 |
| | 1500 pM | 1550 | 1470 | <HIGH> | 1580 |
| | 1500 pM | 1290 | 1370 | 492 | 1440 |
| | 4500 pM | 5770 | 4880 | 4150 | 5100 |
| | 4500 pM | 4170 | 4930 | 8800 | 4000 |
| controls | QC-I | 102 | 139 | 165 | 124 |
| | QC-I | 112 | 167 | 264 | 133 |
| | QC-II | 206 | 323 | 467 | 270 |
| | QC-II | 207 | 277 | 349 | 265 |
| type 1 | 101 | 23.4 | 20.2 | 3.2 | 48.2 |
| type 1 | 101 | 24.4 | 20.5 | 3.36 | 49.1 |
| type 1 | 102 | 7.59 | 16 | 10.4 | 21.7 |
| type 1 | 102 | 8.76 | 14.3 | 9.66 | 19.8 |
| type 1 | 103 | 27.2 | 16.9 | 9.09 | 24.2 |
| type 1 | 103 | 25.5 | 23.6 | 10.3 | 30.7 |
| type 2 | 104 | 414 | 21.1 | 39.8 | 374 |
| type 2 | 104 | 407 | 17.3 | 34.3 | 340 |
| type 2 | 105 | 279 | 16.9 | 16.4 | 106 |
| type 2 | 105 | 320 | 20.2 | 19.4 | 118 |
| type 1 | 106 | 29 | 18.8 | 27.3 | 86.3 |
| type 1 | 106 | 29.6 | 20.8 | 28.5 | 86.8 |
| type 2 | 107 | 300 | 22.4 | 25.2 | 264 |
| type 2 | 107 | 302 | 22.5 | 20.6 | 250 |
| type 2 | 108 | 298 | 21.5 | 5.65 | 267 |
| type 2 | 108 | 387 | 23.8 | 10.2 | 304 |
| type 2 | 109 | <LOW> | 31.1 | 21.6 | 14.6 |
| type 2 | 109 | <LOW> | 23.7 | 26.7 | 17.3 |
| type 1 | 110 | 9.51 | 35.2 | 28.6 | 76.9 |
| type 1 | 110 | 14.3 | 31.7 | 29.2 | 123 |
| type 1 | 111 | 66.3 | 11.3 | 22.4 | 107 |
| type 1 | 111 | 75.8 | 10.6 | 26.1 | 107 |
| type 1 | 112 | 33.7 | 19.6 | 26.7 | 81 |
| type 1 | 112 | 33.7 | 17.6 | 28.4 | 81.2 |
| type 1 | 113 | 17.7 | 16.2 | 25.8 | 64.6 |
| type 1 | 113 | 17.8 | 15.6 | 21.2 | 49.5 |

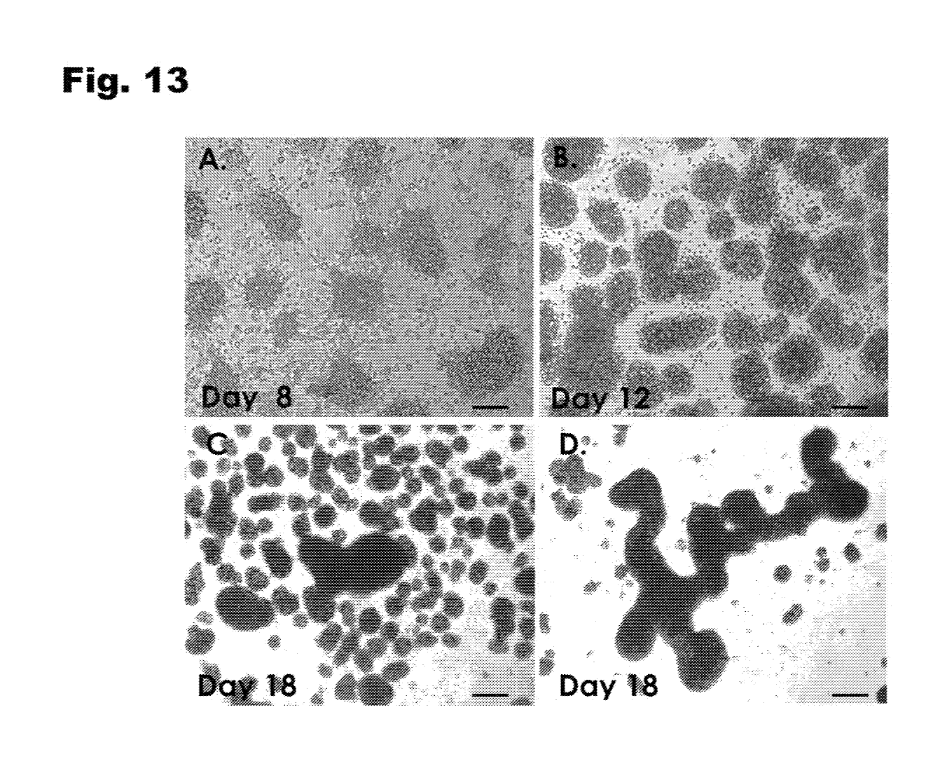

FIG. 13 shows the generation of MDIs from Type 1 subjects. First, MSDCs were generated from PBMCs collected from subjects with type 1 diabetes. After 6 days in de-differentiation medium, MDSCs were treated with pancreatic differentiation medium containing 5 mM glucose. MDSCs formed into MDIs (A). After 6 days in pancreatic medium, MDI aggregates formed into free floating clusters (B). Further treatment of MDI cultures with pancreatic medium containing high glucose (25 mM) led to increases in size and number of MDIs (C and D).

The results above demonstrate that MDIs can be formed from MDSCs isolated from subjects with diabetes.

### EXAMPLE 11

### MDIs Generated from Subjects with Diabetes Express α-Cell and β-Cell Markers

The following demonstrates that MDIs generated from MDSCs isolated from subjects with type 1 or type 2 diabetes express α- and β-cell markers. To examine the functionality of MDIs generated from subjects with type 1 and 2 diabetes, immunofluorescene staining with specific antibodies for β-cell markers (c-peptide and Pdx1) and the α-cell marker (glucagon) was performed.

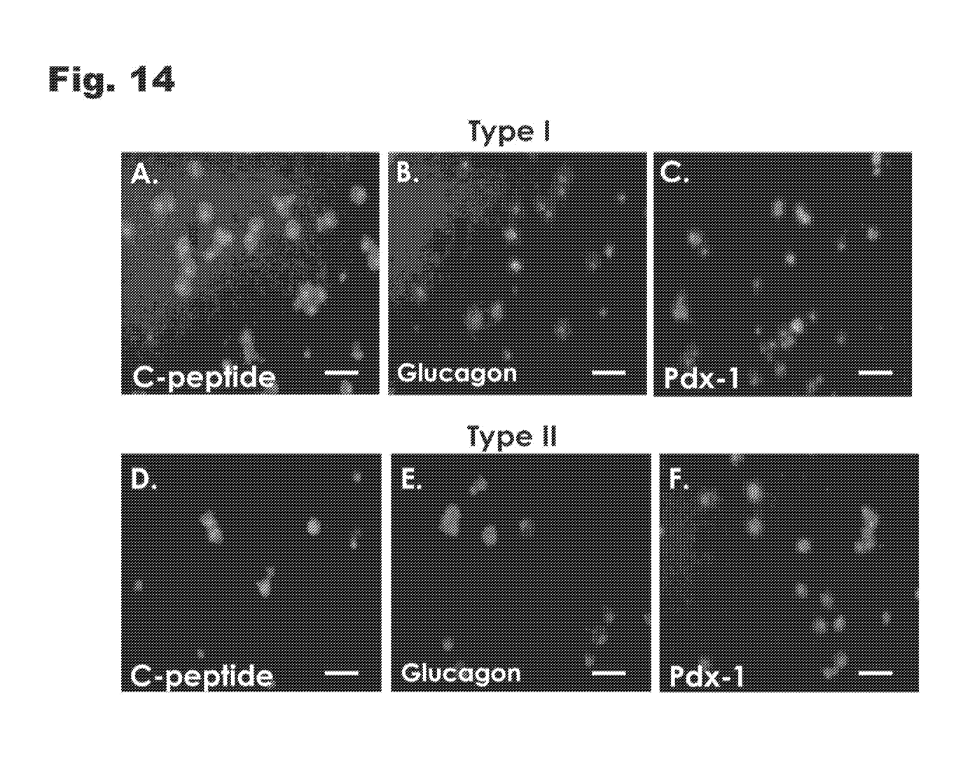

FIG. 14 shows that MDIs derived from subjects with diabetes expressed β-cell markers (c-peptide and Pdx1) and the α-cell marker glucagon. Cytospins were performed on MDIs prior to immunostaining. C-peptide and Pdx1 were detected in approximately 70% cells in both type 1 (A,C) and type 2 (D,F) diabetes. Glucagon staining was observed in approximately 30% of cells in type 1 (B) and type 2 (E).

In addition to expressing α- and β-cell markers, MDIs derived from subjects with diabetes secrete insulin. This was demonstrated by performing ELISA and Luminex assays on both plasma collected from subjects' blood and on the supernatant collected during MDI growth. ELISA assays were performed using either DSL or Mecodia kits following standard operating procedures. Luminex was performed using a Linco diabetes kit containing insulin, c-peptide and glucagon. Each sample was run in triplicate and analyzed against blank and standard controls.

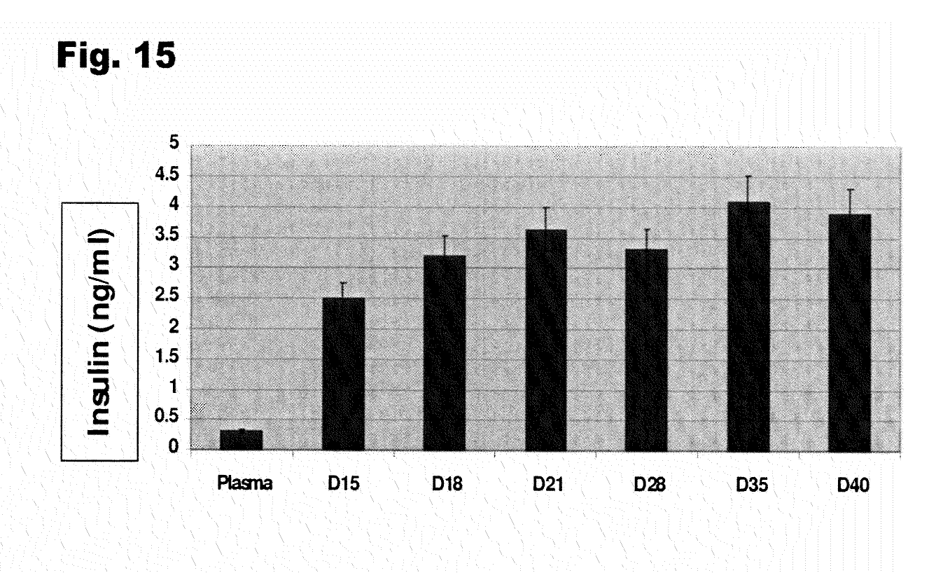

FIG. 15 shows the results of these experiments. ELISA analysis demonstrated that MDIs from subject with diabetes synthesize and secrete insulin (FIG. 15) and c-peptide (not shown) in a glucose-responsive manner. MDIs were cultured for 15 to 40 days in pancreatic differentiation medium containing high glucose, and 1 ml of supernatant was collected and replaced every 3 days. 50 μl of supernatant was used for the ELISA assay and compared to a medium blank and to known concentration standards. An increase in the release of insulin from MDIs ranged from 2.5 (d15) to 4 ng/ml (d35).

Table 6 also shows insulin secretion by MDIs derived from subjects with type I diabetes. An ELISA insulin kit (DSL) was used to measure the amount of insulin secreted by MDIs between days 15 and 40. The level of insulin in the subjects' plasma at the time of collection was also examined.

TABLE 6

| sample | ng/ml |
|---|---|
| Plasma | 0.3 |
| D15 | 2.5 |
| D18 | 3.2 |
| D21 | 3.64 |
| D28 | 3.32 |
| D35 | 4.1 |
| D40 | 3.9 |

The above results demonstrate that MDSCs and MDIs can be generated from subjects with type 1 (n=7) or type 2 (n=7) diabetes. These MDIs express endocrine-specific markers and are able to synthesize and secrete insulin and c-peptide. ELISA and Luminex analysis demonstrated the ability of MDIs from subjects with diabetes to synthesize and secrete insulin and c-peptide in a glucose-responsive manner.

### EXAMPLE 12

### Human Monocyte-Derived Islet Cells can Treat Diabetic Mice

The following demonstrates that MDIs derived from MDSCs isolated from human subjects are capable of treating diabetes in mice. To examine the ability of insulin-producing cells generated in vivo to reverse hyperglycemia, a streptozotocin (STZ)-induced diabetes NOD/SCID mouse model was used.

Hyperglycemia was induced in 8-10 week-old male NOD/SCID mice (Taconic laboratory) by 3 injections of 40 mg/kg of body weight streptozotocin (STZ) that had been freshly dissolved in 0.1 M citrate buffer. Stable hyperglycemia developed between 3-5 days after STZ injections, resulting in blood glucose levels between 300 to 600 mg/dL. Glucose levels in tail vein blood were measured using a glucometer. The animals were grafted with cells or buffer vehicle 48 hours after establishing stable hyperglycemia.

Mice were transplanted with approximately 500 insulin producing clusters (or approximately 1×10⁶ cells in suspension) or 5×10⁶ MDSCs derived from human subjects into the right subcapsular renal space. Blood glucose was then monitored every 2 days for 6-12 weeks after the transplantation. The transplants were excised by unilateral nephrectomy to test for euglycemia reversal, and glucose monitoring was continued. At the end of the experiment, serum was taken from the mice for insulin and c-peptide analysis. Insulin and c-peptide levels were monitored using ELISA and Luminex assays. Concurrent studies were performed on groups of 20 to 40 mice.

Groups A-D were treated as described below (total of 24 mice):

(A) Transplanted mature MDSCs and monitored for 3-12 weeks, transplants were excised, followed by continued glucose monitoring for 2 additional weeks.

(B) Transplanted 500 islet clusters—early-(cultured under high glucose conditions for 3-6 days)(i.e., MDIs at day 15, or “d15”) and monitored for 3-12 weeks, transplants were excised, followed by continued glucose monitoring for 2 additional weeks. The d15 MDIs had been exposed to high glucose conditions for 3 days and exhibited an increase in the expression of PDX1, somatostatin and ngn3. The d15 MDIs also expressed a low level of insulin. The clusters also had an increased rate of proliferation. The size of the d 15 MDIs was 100 to 300 microns. In addition the total number of d15 MDIs in a well of a 6 well plate was 100 to 500 clusters.

(C) Transplanted 500 islet clusters—late-(cultured under high glucose conditions for 7-12 days)(i.e., MDIs at d23) and monitored for 3-12 weeks, transplants were excised, followed by continued glucose monitoring for 2 additional weeks. The d23 MDIs had been exposed to high glucose for 11 days and exhibited a increased level of insulin (2-8 ng/ml) per well of 6 well plate. By immunofluorescene the d23 MDIs exhibited expression of insulin, glucagon and somatostatin within the clusters. The proliferation rate of d23 MDIs was relatively unchanged compared to d15 MDIs. The size of the d23 MDIs was 200-1000 microns. The total number of d23 MDIs in a well of a 6 well plate was 200-1000 clusters.

(D) Sham transplant of krebs-ringer bicarbonate buffer saline without Ca₂ (Vehicle control) injection monitored for 6 weeks, transplants were excised, followed by continued glucose monitoring for 2 additional weeks

MDSCs were generated from buffy coats obtained from a Regional Blood Bank from healthy human donors following standard operating procedures. These samples were screened by the blood center prior to shipment. The samples were processed via a common lymphocyte separation method in which the mononuclear fraction was collected, washed and counted using a Vi-cell particle counter as previously described. MDSCs were prepared from PBMCs as described above.

PBMCs collected from the mononuclear fractions were then resuspended in medium and seeded onto treated tissue culture dishes. The cells were then incubated at 37° C. in 5% CO₂. When MDSCs were fully developed, a subset was harvested and prepared for control injections.

To generate MDIs, MDSCs were further grown in de-differentiation medium for 6 days. MDSCs were then washed and fed with a pancreatic medium containing low glucose (5 mM) for 6 days. Next, cultures were treated with pancreatic medium containing high glucose (25 mM). MDIs were then incubated at 37° C. in 5% CO, for a either 3 days or 11 days before harvesting. MDIs were harvested by placing them in a falcon tube, followed by centrifugation at 500 rpm for 5 minutes. The medium was then removed and replaced with pancreatic medium. Cells were stored at 37° C. until injection.

Prior to injection into NOD/SCID mice, MDIs were centrifuged at 500 rpm for 5 minutes and washed in fresh pancreatic medium. The cells were then centrifuged again as described above and resuspended in 50 μl pancreatic medium. Next, the cells were collected into a small gauge needle and injected through the kidney into the kidney capsule. All mouse surgeries were performed following approved animal protocols under sterile conditions.

Prior to injection into mouse kidney capsules, MDSCs and islet-like clusters were characterized by flow cytometry, immunohistochemistry and Real Time PCR. The phenotype of MDSCs was determined by using endocrine-specific markers which included insulin, c-peptide, somatostatin and glucagon. To test the functionality of MDIs, the expression of insulin, c-peptide, glucagon, and somatostatin were examined both by immunohistochemistry and Real Time PCR.

For PCR-based characterization, total RNA was extracted from both MDSCs and MDIs, and cDNA synthesized using standard protocols. To determine the relative expression of several pancreatic genes, Sybr green and/or Taqman Real Time PCR assays were used. All samples are compared to GADPH and B-actin standards to determine the relative gene expression.

Following injection of MDSC control cells, saline control, or d15 or d23 MDIs into STZ-induced hyperglycemic NOD/SCID mouse kidney capsules, blood glucose levels were monitored over 60 days. The ability of early MDIs (d15) were compared to late MDIs (d23) in lowering blood glucose levels.

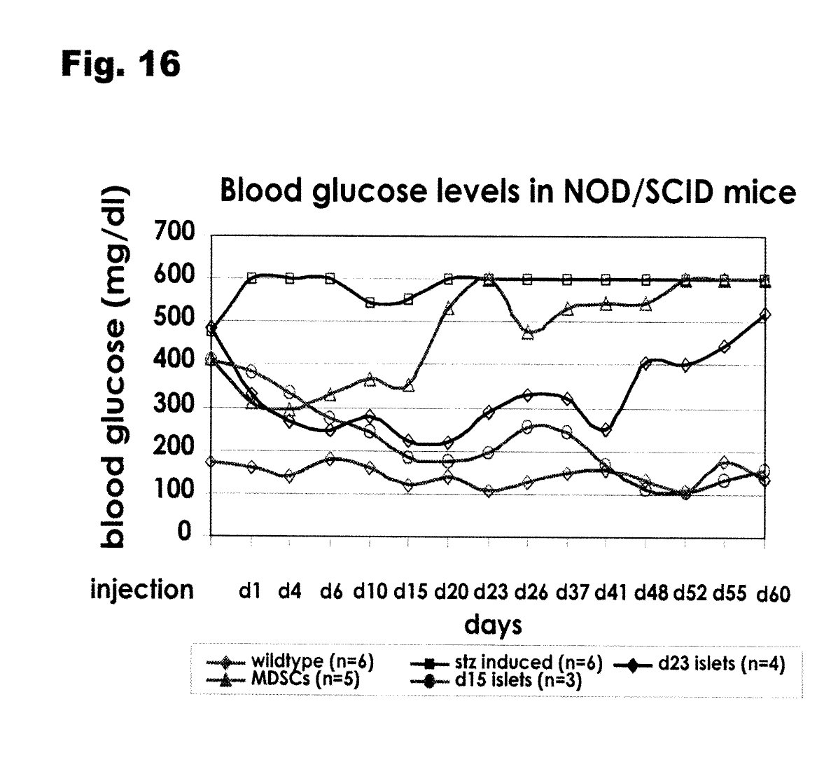

FIG. 16 shows the results of these experiments. Blood glucose levels of wildtype mice were approximately 150-200 mg/dl, while those of STZ-induced NOD/SCID mice were elevated to around 600 mg/dl. STZ-induced hyperglycemic NOD/SCID mice injected with d15 MDIs showed blood glucose levels approaching wildtype, as did mice injected with d23 MDIs. However mice injected with d23 MDIs showed elevated blood glucose levels after 6-7 weeks.

Table 7 also shows the results of measuring blood glucose levels in wildtype and STZ-induced NOD/SCID mice injected with saline control, MDSCs, or d15 or d23 MDIs.

TABLE 7

| | injection | d1 | d4 | d6 | d10 | d15 | d20 | d23 | d26 | d37 | d41 | d48 | d52 | d55 | d60 |
|---|---|---|---|---|---|---|---|---|---|---|---|---|---|---|---|
| wildtype (n = 6) | 170 | 160 | 139 | 178 | 158 | 121 | 139 | 108 | 128 | 146 | 154 | 130 | 108 | 174 | 137 |
| stz induced (n = 6 | 479 | 601 | 601 | 601 | 543 | 553 | 601 | 601 | 601 | 601 | 601 | 601 | 601 | 601 | 601 |
| d23 islets (n = 4) | 486 | 331 | 268 | 245 | 277 | 221 | 221 | 289 | 332 | 322 | 252 | 404 | 400.5 | 446 | 521 |
| MDSCs (n = 5) | 412 | 312 | 295 | 330 | 364 | 354 | 534 | 601 | 476 | 534 | 546 | 544 | 601 | 601 | 601 |
| d15 islets (n = 3) | 409 | 383 | 336 | 274 | 241 | 182 | 174 | 196 | 256 | 243 | 167 | 110 | 104 | 132 | 155 |

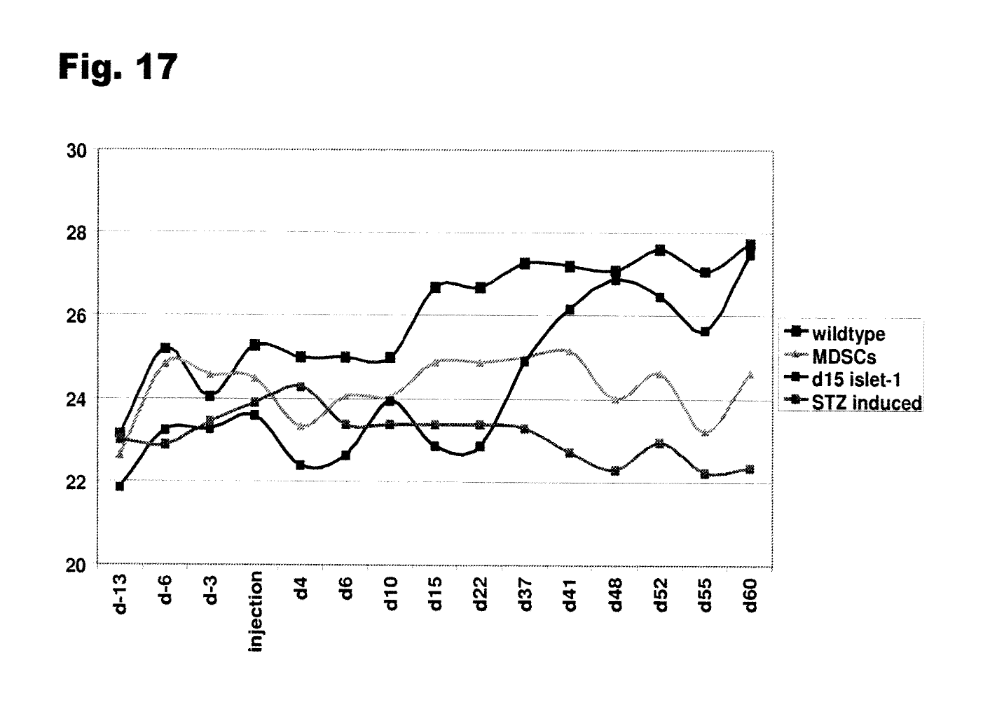

Body weights of STZ-induced hyperglycemic NOD/SCID mice transplanted with day 15 MDIs were also examined for 73 days, and compared to wildtype, and STZ-induced hyperglycemic NOD/SCID mice injected with either saline control. MDSCs, or d15 MDIs. The results of these experiments are shown in FIG. 17 and Table 8. Body weights of wildtype mice gradually increased from 24 to 28 grams. STZ-induced mice exhibited a decrease from 24 to 22 grams. Mice injected with d15 MDIs exhibited an increase in the overall body weight beginning at 21 days post transplant. However mice injected with MDSCs failed to increase and gradually reduced over time to STZ induced levels.

TABLE 8

| | d-13 | d-6 | d-3 | injection | d4 | d5 | d10 | d15 | d22 | d37 | d41 | d48 | d52 | d55 | d [symbol] |
|---|---|---|---|---|---|---|---|---|---|---|---|---|---|---|---|
| wildtype | 23.155 | 25.2 | 24.05 | 25.3 | 25 | 25 | 25 | 26.705 | 26.705 | 27.305 | 27.215 | 27.1 | 27.625 | 27.08 | [symbol] |
| MDSCs | 22.6 | 24.8 | 24.6 | 24.5 | 23.4 | 24.1 | 24.1 | 24.9 | 24.9 | 25.0 | 25.2 | 24.0 | 24.6 | 23.2 | [symbol] |
| d15 islet | 21.9 | 23.3 | 23.3 | 23.6 | 22.4 | 22.6 | 23.9 | 22.87 | 22.87 | 24.9 | 26.2 | 26.9 | 26.5 | 25.7 | [symbol] |
| [symbol] Z induced | 23.012 | 22.9 | 23.5 | 23.9 | 24.3 | 23.4 | 23.4 | 23.4 | 23.4 | 23.3 | 22.73 | 22.32 | 22.97 | 22.24 | [symbol] |

[symbol]

indicates data missing or illegible when filed

α-(glucagon) and β-cell (insulin) marker expression was also examined n STZ-induced hyperglycemic NOD/SCID mice transplanted with day 15 MDIs. Kidneys from NOD/SCID mice injected with d15 MDIs were collected within an hour of injection and fixed in 10% formalin overnight, and then processed in paraplast. Tissues were then sectioned and stained with antibodies for insulin and glucagon.

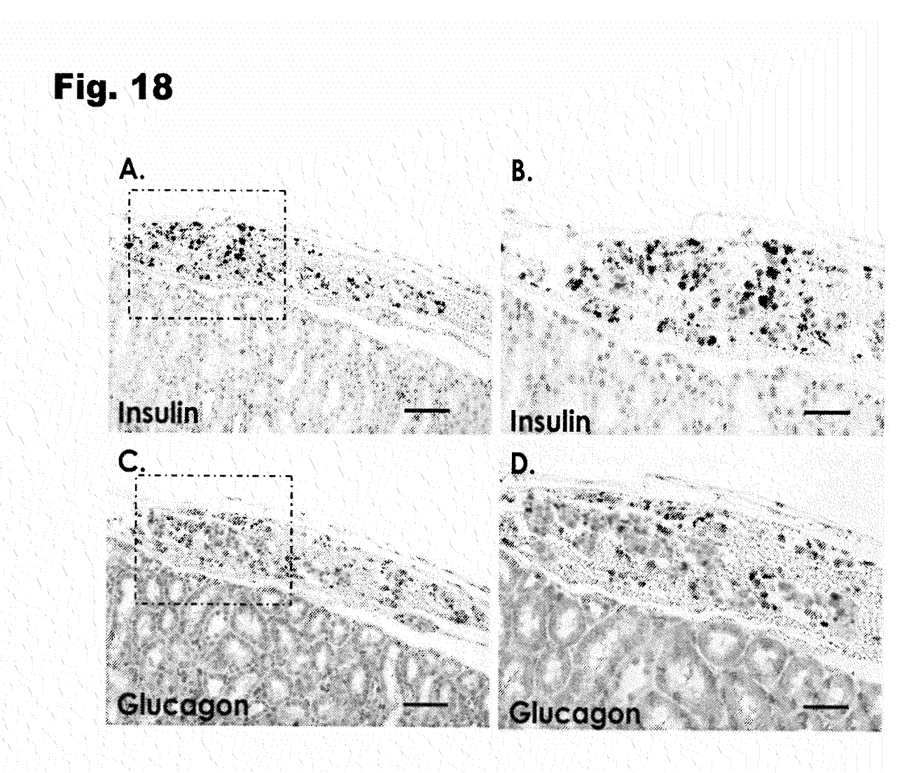

FIG. 18 shows the results of these experiments. Insulin (A,B) and glucagon (C,D) staining was observed in MDIs injected under the kidney capsule, indicating that the MDIs comprised both α- and β-cells.

Expression of α- and β-cell markers were also analyzed in plasma from the above-described NOD/SCID mice. Plasma was collected from untreated control mice, and from STZ-induced hyperglycemic NOD/SCID mice that were injected with MDSCs or MDIs. The results of these experiments is shown in Table 9. An increase in the level of human glucagon in mice #134 and #145 was observed. Both mice were injected with MDIs. Mouse #134 had been injected with early islets and #145 with late islets. Both mice exhibited a decrease in blood glucose levels. No change in glucagon levels were observed for untreated or MDSC-injected mice.

TABLE 9

| | | C-peptide (pM) | Glucagon (pM) | Insulin (pM) |
|---|---|---|---|---|
| Standards | 6.2 pM | 6.47 | 6.33 | 7.12 |
| | 6.2 pM | 5.88 | 3.94 | 4.73 |
| | 18.5 pM | 20.5 | 23.8 | 17.7 |
| | 18.5 pM | 17.1 | 16.5 | 21.9 |
| | 55.6 pM | 58.1 | 60.1 | 59.7 |
| | 55.6 pM | 50.4 | 49.3 | 46.2 |
| | 166.7 pM | 162 | 164 | 176 |
| | 166.7 pM | 184 | 170 | 171 |
| | 500 pM | 501 | 441 | 501 |
| | 500 pM | 502 | 576 | 463 |
| | 1500 pM | 1610 | <HIGH> | 1700 |
| | 1500 pM | 1740 | 994 | 1530 |
| | 4500 pM | 3120 | 465 | 3390 |
| | 4500 pM | 4380 | <HIGH> | 5320 |
| controls | QC-1 | 101 | 164 | 121 |
| | QC-1 | 108 | 195 | 126 |
| | QC-2 | 292 | 622 | 309 |
| | QC-2 | 271 | 313 | 295 |
| Mice plasma | 134 | <LOW> | 10.4 | <LOW> |
| | 134 | <LOW> | 10.4 | <LOW> |
| | 140 | <LOW> | 0.811 | <LOW> |
| | 140 | <LOW> | 0.707 | <LOW> |
| | 141 | <LOW> | <LOW> | <LOW> |
| | 141 | <LOW> | <LOW> | <LOW> |
| | 143 | <LOW> | <LOW> | <LOW> |
| | 143 | <LOW> | <LOW> | <LOW> |
| | 144 | <LOW> | <LOW> | <LOW> |
| | 144 | <LOW> | <LOW> | <LOW> |
| | 145L | <LOW> | 43 | 3.17 |
| | 145L | <LOW> | 31.5 | <LOW> |
| | 146 | <LOW> | 2.78 | <LOW> |
| | 146 | <LOW> | 2.35 | <LOW> |
| | 147 | <LOW> | <LOW> | <LOW> |
| | 147 | <LOW> | <LOW> | <LOW> |
| | 148 | <LOW> | <LOW> | <LOW> |
| | 148 | <LOW> | <LOW> | <LOW> |
| | 149 | <LOW> | <LOW> | <LOW> |
| | 149 | <LOW> | <LOW> | <LOW> |
| | 151 | <LOW> | <LOW> | <LOW> |
| | 151 | <LOW> | <LOW> | <LOW> |
| | 153 | <LOW> | <LOW> | <LOW> |
| | 153 | <LOW> | <LOW> | <LOW> |

The experiments described above demonstrate that MDSCs have no effect on blood glucose levels. Furthermore, d15 MDIs injected into STZ-induced NOD/SCID hyperglycemic mice are capable of reducing blood glucose levels to near-normal levels for a prolonged period of time, and restoring body weight to normal range. d23 MDIs also lower blood glucose levels to 300 mg/dL compared to levels of over 500 mg/dL in STZ-induced mice. However d23 MDIs were only effective for 6 weeks, after which mice returned to a diabetic state.

The above experiments are consistent with early (d15) MDIs being capable of proliferating or renewal within the kidney capsule, although the more terminally differentiated late (d23) MDIs have limited proliferation. In addition, an increase in the secretion of human glucagon was observed in STZ-induced NOD/SCID mice that were injected with MDIs, and these mice had lower blood glucose levels. The level of glucagon detected in NOD/SCID mice transplanted with MDIs was within human physiological ranges.

The results described above demonstrate that MDIs generated from human MDSCs are capable of treating symptoms of diabetes, including elevated blood glucose levels.

What is claimed is:

1. A method of generating a MDI, the method comprising
(a) providing a composition comprising a stem cell;
(b) contacting the stem cell with at least one differentiation factor wherein the differentiation factor induces differentiation of the stem cell into a MDI.

2. The method of claim 1, wherein the stem cell is derived from a subject.

3. The method of claim 1 wherein the stem cell is derived from a monocyte.

4. The method of claim 1 wherein the stem cell is a MDSC.

5. The method of claim 1, further comprising:
(a) contacting the cell with a low concentration of glucose; and
(b) contacting the cell with a high concentration of glucose.

6. The method of claim 4 wherein the low concentration of glucose is 2-15 mM

7. The method of claim 6 wherein the low concentration of glucose is 5 mM

8. The method of claim 4 wherein the high concentration of glucose is 5-40 mM

9. The method of claim 7 wherein the high concentration of glucose is 25 mM

10. The method of claim 1 wherein the

11. The method of claim 2, wherein the subject has either type 1 or type 2 diabetes.

12. A method of treating diabetes, the method comprising administering to a patient in need thereof the MDI of claim 2.

13. The method of claim 10, wherein the subject is the same individual as the patient.

14. An isolated MDI, wherein the MDI secretes insulin in the presence of glucose.

15. The MDI of claim 14, wherein the MDI is derived from an MDSC.

16. A composition comprising a plurality of MDI according to claim 14, wherein the composition comprises a α-cell, β-cell, γ-cell, δ-cell or combination thereof.

17. An isolated MDI made by the method of claim 1.


SEQUENCE LISTING (28 sequences)

SEQ ID NO: 1 - DNA, 24 bp, Artificial Sequence
  primer
gatgaagtaccccaacctgtttac

SEQ ID NO: 2 - DNA, 24 bp, Artificial Sequence
  primer
aagttctctttccaatttcaccac

SEQ ID NO: 3 - DNA, 24 bp, Artificial Sequence
  primer
tcacctttgaacttcgagatacag

SEQ ID NO: 4 - DNA, 24 bp, Artificial Sequence
  primer
ccagaagcttaaaagaaagattgg

SEQ ID NO: 5 - DNA, 22 bp, Artificial Sequence
  primer
gggcaagttcttccaatatgac

SEQ ID NO: 6 - DNA, 21 bp, Artificial Sequence
  primer
gtcttgggtgggtagagcaat

SEQ ID NO: 7 - DNA, 19 bp, Artificial Sequence
  primer
acaagcagccggagaagac

SEQ ID NO: 8 - DNA, 21 bp, Artificial Sequence
  primer
ctgctggagttgcttcatcat

SEQ ID NO: 9 - DNA, 20 bp, Artificial Sequence
  primer
gttccactggatgaccgaaa

SEQ ID NO: 10 - DNA, 20 bp, Artificial Sequence
  primer
tcattccaccaactgcaaag

SEQ ID NO: 11 - DNA, 20 bp, Artificial Sequence
  primer
tggcacaggtttaagaacga

SEQ ID NO: 12 - DNA, 20 bp, Artificial Sequence
  primer
gtcaggctggtctcgaactc

SEQ ID NO: 13 - DNA, 20 bp, Artificial Sequence
  primer
agctttacaaggacccatgc

SEQ ID NO: 14 - DNA, 18 bp, Artificial Sequence
  primer
cctcgtacggggagatgt

SEQ ID NO: 15 - DNA, 18 bp, Artificial Sequence
  primer
gaggggtccctgcagaag

SEQ ID NO: 16 - DNA, 20 bp, Artificial Sequence
  primer
ggttcaagggctttattcca

SEQ ID NO: 17 - DNA, 22 bp, Artificial Sequence
  primer
aacgaggcttcttctacacacc

SEQ ID NO: 18 - DNA, 24 bp, Artificial Sequence
  primer
ctgcgtctagttgcagtagttctc

SEQ ID NO: 19 - DNA, 20 bp, Artificial Sequence
  primer
agctgctgtctgaacccaac

SEQ ID NO: 20 - DNA, 21 bp, Artificial Sequence
  primer
agaaattcttgcagccagctt

SEQ ID NO: 21 - DNA, 21 bp, Artificial Sequence
  primer
atttccaacttggggatgttt

SEQ ID NO: 22 - DNA, 22 bp, Artificial Sequence
  primer
tttaagaaacctggttgccagt

SEQ ID NO: 23 - DNA, 21 bp, Artificial Sequence
  primer
aatcgaatgcacaacctcaac

SEQ ID NO: 24 - DNA, 21 bp, Artificial Sequence
  primer
gtacaagctgtggtccgctat

SEQ ID NO: 25 - DNA, 20 bp, Artificial Sequence
  primer
caaagttgtcatggatgacc

SEQ ID NO: 26 - DNA, 17 bp, Artificial Sequence
  primer
ccatggagaaggctggg

SEQ ID NO: 27 - DNA, 20 bp, Artificial Sequence
  primer
gcttgctgatccacatctgc

SEQ ID NO: 28 - DNA, 19 bp, Artificial Sequence
  primer
tggacatccgcaaagacct